(19) United States
(12) Patent Application Publication
(10) Pub. No.: US 2016/0000385 A1
(43) Pub. Date: Jan. 7, 2016

(54) DETERMINING INFORMATION FLOW USING PHYSIOLOGICAL PARAMETERS

(71) Applicant: Google Inc., Mountain View, CA (US)

(72) Inventors: Morten Just Petersen, San Francisco, CA (US); Joao Paulo Gil de Paiva, Zurich (CH); Emmet Connolly, Mountain View, CA (US); Tim Carlo Jorges, Zurich (CH)

(21) Appl. No.: 14/851,620

(22) Filed: Sep. 11, 2015

Related U.S. Application Data
(63) Continuation of application No. 14/147,323, filed on Jan. 3, 2014.

(51) Int. Cl. A61B 5/00; A61B 5/0205; A61B 5/0402

(52) U.S. Cl. CPC A61B 5/7425; A61B 5/0402; A61B 5/0205; A61B 5/681; A61B 5/0022; A61B 5/7475; A61B 5/725; A61B 5/7282; A61B 5/7405; A61B 5/7455; A61B 5/0533

(57) ABSTRACT

Systems, devices, and processes may be related to determining the flow of information based on received physiological parameters. In one example, a computing device includes at least one processor and at least one module operable by the at least one processor to receive an indication of a physiological parameter representative of a physiological condition of a user and receive a request to output notification information. The at least one module may also be operable by the at least one processor to determine, based on the physiological parameter, whether to output the notification information and selectively output, based on the determination, the notification information.

This application is a continuation of U.S. patent application Ser. No. 14/147,323, titled “DETERMINING INFORMATION FLOW USING PHYSIOLOGICAL PARAMETERS” and filed Jan. 3, 2014, the entire contents of which are incorporated herein by reference.

### BACKGROUND

A computing device (e.g., a mobile phone, a wearable computing device, etc.) may receive notification information associated with a user. For instance, a computing device may receive notification information indicating that a new instant message was received by an instant messaging account associated with the user. To alert the user to the receipt of the notification information and the new instant message indicated by the notification information, the computing device may output a visual, audible, and/or haptic type alert that indicates to the user that the new instant message was received.

### SUMMARY

In one example, the disclosure is directed to a method that includes receiving, by a computing device, an indication of a physiological parameter representative of a physiological condition of a user, receiving, by the computing device, a request to output notification information, determining, by the computing device and based on the physiological parameter, whether to output the notification information, and selectively outputting, by the computing device and based on the determination, the notification information.

In another example, the disclosure is directed to a computing device that includes at least one processor and at least one module operable by the at least one processor to receive an indication of a physiological parameter representative of a physiological condition of a user, receive a request to output a notification information, determine, based on the physiological parameter, whether to output the notification information, and selectively output, based on the determination, the notification information.

In another example, the disclosure is directed to a computer-readable storage medium comprising instructions that, when executed, cause one or more processors of a computing device to receive an indication of a physiological parameter representative of a physiological condition of a user, receive a request to output a notification information, determine, based on the physiological parameter, whether to output the notification information, and selectively output, based on the determination, the notification information.

The details of one or more examples are set forth in the accompanying drawings and the description below. Other features, objects, and advantages of the disclosure will be apparent from the description and drawings, and from the claims.

### BRIEF DESCRIPTION OF DRAWINGS

FIG. 1 is a conceptual diagram illustrating an example computing system that includes an information server system that outputs notification information to an example computing device, in accordance with one or more aspects of the present disclosure.

FIG. 2 is a conceptual diagram illustrating an example computing system that includes a mobile computing device that output notification information to an example computing device, in accordance with one or more aspects of the present disclosure.

FIG. 3 is a block diagram illustrating an example computing device configured to manage notification information based on one or more physiological parameters.

FIG. 4 is a block diagram illustrating an example information server system configured to output notification information.

FIG. 5 is a block diagram illustrating an example mobile computing device configured to output notification information.

FIG. 6 is a block diagram illustrating an example computing device that outputs graphical content for display at a remote device, in accordance with one or more techniques of the present disclosure.

FIG. 7 is a flowchart illustrating example operations of a computing device configured to determine whether to output notification information.

FIG. 8 is a flowchart illustrating example operations of a computing device configured to withhold output of notification information when a value of a physiological parameter exceeds a threshold value.

FIG. 9 is a flowchart illustrating example operations of a computing device configured to filter a plurality of items based on a physiological parameter.

### DETAILED DESCRIPTION

In general, techniques of this disclosure may enable a computing device (e.g., a wearable computing device, mobile computing device, networked server, etc.) to automatically manage information flow based on one or more physiological parameters representative of a physiological condition of a user. Typically, a computing device may perform a variety of different tasks (e.g., output notifications or alerts indicative of notifications, output search results, present a list of executable application, etc.) for delivering respective information to the user. These different tasks may be performed without regard to the condition of the user. However, the user may be nervous, stressed, excited, calm, concentrated, or otherwise experiencing a particular physiological condition that may be related to a respective situation or event experienced by the user. And, the user may not be interested in certain information during one or more of these physiological conditions. Therefore, the computing device may output, for delivery to the user, information that may not be optimized for the condition of the user.

As described herein, one or more computing devices may determine when to output information, determine the content of the information, or otherwise manage how information is delivered to a user based on one or more physiological parameters detected from the user. In this manner, the computing device may be configured to determine a condition of the user and output information relevant to that determined condition. For example, a computing device may withhold at least some notification information based on a received physiological parameter. The computing device may be configured to receive an indication of a physiological parameter representative of a physiological condition of a user. The physiological parameter may be representative of a galvanic skin response (GSR), a pulse rate, a breathing rate, or an electrogram of the user. In some examples, one or more sensors of a wearable computing device (e.g., a watch, a wristband, a smartwatch, a chest strap, smart eye glasses, or any other such devices) may generate a signal indicative of the physiological parameter.

The computing device may be configured to receive a request to output notification information. This request may be from an application executing on the computing device or from an application or service executing on another device associated with the user. The notification information may include a representation of the notification (e.g., a brief description of a communication message or event) and/or an indication of the notification (e.g., an visual, audible, or tactile alert that indicates the presence of the notification). The computing device may then determine, based on the received physiological parameter, whether to output the notification information and selectively output, based on the determination, the notification information. In this manner, the computing device may be configured to withhold notification information in response to determining that one or more physiological parameters indicate that the notification information should be withheld instead of output for delivery to the user (e.g., via some output device such as a display device, speaker, visual indicator, or haptic device).

In other examples, a computing device may be configured to manage the flow of information by filtering items for presentation to the user. The computing device may be configured to receive an indication of a physiological parameter representative of a physiological condition of a user and receive, for display at a display device associated with a wearable computing device, a plurality of items. The plurality of items may be the result of a search request (e.g., an list of actionable web links, photographs, and/or videos resulting from an internet search query), a plurality of available applications executable by the computing device or the wearable computing device, a list of destinations, a list of events, a group of communications, or any other such items that may be delivered to the user. The computing device, or another device, may generate the plurality of items in response to a user request or predictively based on one or more factors.

The computing device may then filter, based on one or more received physiological parameters, the plurality of items to generate a subset of the plurality of items and output, for display at the display device associated with the wearable computing device, the subset of the plurality of items. In this manner, the computing device may be configured to deliver search results, executable applications, photographs, videos, communications, or any other information appropriate for the physiological condition, and perhaps current situation, of the user. In some examples, the computing device may filter the plurality of items based on the physiological conditions and one or more user-selectable thresholds.

Throughout the disclosure, examples are described in which a computing device and/or a computing system may analyze information (e.g., physiological parameters, thresholds, notification information, other items, etc.) associated with a computing device only if the computing device receives permission from the user to analyze the information. For example, in situations discussed below in which the computing device may collect or may make use of information associated with the user, the user may be provided with an opportunity to provide input to control whether programs or features of the computing device can collect and make use of user information (e.g., information about a user's current physiological parameters, location, active applications, etc.), or to dictate whether and/or how to the computing device may receive content that may be relevant to the user. In addition, certain data may be treated in one or more ways before it is stored or used by the computing device and/or computing system, so that personally-identifiable information is removed. For example, a user's identity may be treated so that no personally identifiable information can be determined about the user, or a user's geographic location may be generalized where location information is obtained (such as to a city, ZIP code, or state level), so that a particular location of a user cannot be determined. Thus, the user may have control over how information about the user is collected and used by the computing device.

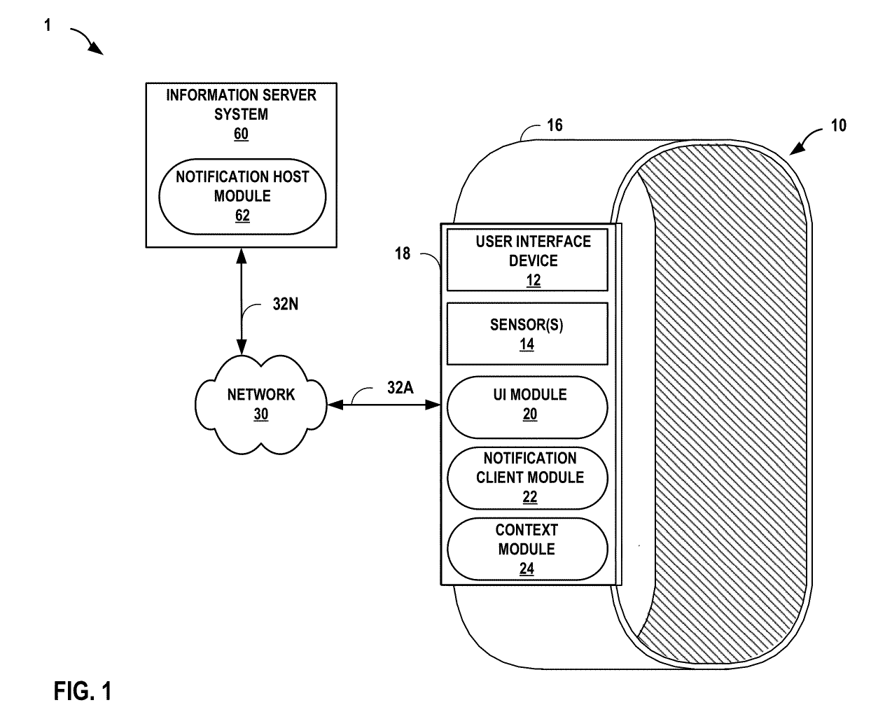

FIG. 1 is a conceptual diagram illustrating an example computing system 1 that includes an information server system 60 that outputs notification information to an example computing device 10. Computing system 1 of FIG. 1 is an example computing system that includes computing device 10, information server system 60, and network 30, that may perform various tasks described herein.

Network 30 may include any public or private communication network, for instance, a cellular network, Wi-Fi® network, and/or other type of network for transmitting data between two or more computing devices. Computing device 10 and information server system 60 may send and receive data across network 30 using any suitable communication techniques known in the art. For example, computing device 10 may be operatively coupled to network 30 using network link 32A and information server system 60 may be operatively coupled to network 30 by network link 32N. Network links 32A and 32N may utilize the same or different communication protocols. Network 30 may include network hubs, network switches, network routers, etc., that are operatively inter-coupled thereby providing for the exchange of information between computing device 10 and information server system 60. In some examples, network links 32A and 32N (collectively, “network links 32”) may be Ethernet, ATM, or other network connections. Such connections may be wireless and/or wired connections.

Information server system 60 may include any suitable remote computing system, such as one or more desktop computers, laptop computers, mainframes, servers, cloud computing systems, etc. capable of sending and receiving information (e.g., notification information) across network link 32N to network 30. In some examples, information server system 60 may include a host server for one or more notification system services. One or more computing devices, such as computing device 10, may access a notification service hosted by information server system 60 for transmitting and/or receiving notification information between platforms, applications, and services executing at the one or more computing devices. In some examples, information server system 60 may include a cloud computing system that provides notification services through network 30 to the one or more computing devices that access the notification services via access to the cloud provided by information server system 60.

In the example of FIG. 1, information server system 60 includes notification host module 62. Module 62 may perform operations described herein using software, hardware, firmware, or a mixture of hardware, software, and/or firmware residing in and/or executing at information server system 60. In some examples, information server system 60 may execute module 62 with one or more processors or one or more devices. Information server system 60 may execute module 62 as a virtual machine executing on underlying hardware. Module 62 may execute as one or more services of an operating system or computing platform. Module 62 may execute as one or more executable programs at an application layer of a computing platform.

Notification host module 62 may perform functions for routing notification information between one or more computing devices, such as computing device 10, over network 30. Notification host module 62 may perform functions for hosting a notification service and outputting notification information for transmission to one or more computing devices, including computing device 10. For example, notification host module 62 may receive notification information that indicates a message was received by an instant messaging account associated with computing device 10 and may output the notification information for transmission across network 30 to computing device 10. In other examples, notification host module 62 may be configured to generate and transmit requests to output notification information related to any type of communication message that has been received, any type of upcoming event, a status update for one or more applications, one or more system conditions, or any other feature for which a notification may be delivered to a user regarding an item for the user. Information server system 60 and notification host module 62 may operate as a networked push notification system (e.g., a cloud-based push notification system) configured to push, or transmit, notification information to another computing device (e.g., computing device 10) indicative of an event that occurred remote from computing device 10 (e.g., an application or services executing on information server system 60 or another networked device). System 1 may also operate, in addition or alternative to a push notification system, as a pull notification system in which notification client module 22 of computing device 10 requests, or pulls, notification information from notification host module 62 of notification server system 60. Any system or device described herein may operate as at least part of a push and/or pull notification system.

As described herein, notification information may include any type of information related to a notification. For example, notification information may include a representation of the notification, wherein the representation includes a graphical representation of the notification. The graphical representation of the notification may be a short message relaying a synopsis or portion of the message to which the notification is directed. Notification information may additionally, or alternatively, include an indication of the notification which may be an alert regarding the presence of the notification. The indication of the notification may be a visual alert (e.g., activation of one or more lights), an audible alert (e.g., a generated sound), or a tactile alert (e.g., activation of a haptic device), as examples. In some examples, the notification information may include a representation of the notification and one or more indications of the notification for delivery to the user.

As described herein, computing device 10 may be configured to manage or control the delivery of information to a user (e.g., a user of computing device 10). For example, computing device 10 may be configured to determine whether to output some or all of notification information received from information server system 60 in a request to output the notification information. Computing device 10 may receive physiological parameters regarding the physical condition of the user of computing device 10. One or more sensors may generate physiological parameters, or values of physiological parameters, from detectable physiological signals from the user. For example, physiological parameters may be representative of a galvanic skin response, a breathing rate, a pulse rate, a temperature, an electrogram, or any other detectable signal or physiological state. Computing device 10 may include one or more of the sensors (e.g., within housing 18 of computing device 10 or attached to housing 18) or be in wired or wireless communication with one or more remote sensors associated with the user.

One or more of the physiological parameters may be representative of a physiological condition of the user. The physiological condition may be a stressed condition, an excited condition, a concentrated condition, a calm condition, a happy condition, a tense condition, or any other type of condition. Each of these conditions may be associated with a type of situation or event being experienced by the user. Certain information may be useful in these situations or events while other information may be distracting or unwanted during these situations or events. Therefore, computing device 10 may predict which information may be useful and which information may be unwanted during any period of time based on one or more physiological parameters of the user. For example, computing device 10 may compare values of one or more physiological parameters to respective thresholds or other rules and determine, based on the comparison, which notification information to output for delivery to the user. In this manner, computing device 10 may withhold certain notification information from delivery to the user in response to determining that a value of a physiological parameter exceeds a threshold instead of delivering the notification information. Computing device 10 may also output, for delivery to the user, previously withheld notification information in response to determining that a value of the physiological parameter no longer exceeds the threshold.

As shown in the example of FIG. 1, computing device 10 is a wearable computing device (e.g., a computerized watch, computerized eyewear, computerized headwear, computerized gloves, etc.). However, in other examples, computing device 10 may be a tablet computer, a mobile phone, a personal digital assistant (PDA), a laptop computer, a gaming system, a media player, an e-book reader, a television platform, an automobile navigation system, or any other type of mobile and/or non-mobile computing device that is configured to receive notification information from information server system 60 and determine whether to output, based on one or more physiological parameters, notification information (e.g., a representation of the notification or an indication of the notification) for presentation to the user associated with computing device 10. If computing device 10 is not a wearable computing device, computing device 10 may receive physiological parameters from one or more remote sensors associated with the user.

In some examples, computing device 10 may include attachment mechanism 16 and electrical housing 18. Housing 18 of computing device 10 includes a physical portion of a wearable computing device that houses a combination of hardware, software, firmware, and other electrical components of computing device 10. For example, FIG. 1 shows that within housing 18, computing device 10 may include a user interface device (UID) 12, one or more sensors 14, user interface (UI) module 20, notification client module 22, and context module 24. Modules 20, 22, and 24 may perform operations described herein using software, hardware, firmware, or a mixture of hardware, software, and/or firmware residing in and/or executing at computing device 10. Computing device 10 may execute modules 20, 22, and 24 with one or more processors located within housing 18. In some examples, computing device 10 may execute modules 20, 22, and 24 as one or more virtual machines executing on underlying hardware of computing device 10 located within housing 18. Modules 20, 22, and 24 may execute as one or more services or components of operating systems or computing platforms of computing device 10. Modules 20, 22, and 24 may execute as one or more executable programs at application layers of computing platforms of computing device 10. In other examples, UID 12, one or more sensors 14, and/or modules 20, 22, and 24 may be arranged remotely to housing 18 and be remotely accessible to computing device 10, for instance, via interaction by computing device 10 with one or more network services operating at network 30 in a network cloud or direct device-to-device communication.

Attachment mechanism 16 may include a physical portion of a wearable computing device that comes in contact with a body (e.g., tissue, muscle, skin, hair, clothing, etc.) of a user when the user is wearing the computing device. For example, in cases where computing device 10 is a watch, attachment mechanism 16 may fit around a user's wrist and come in contact with the skin of the user, when computing device 10 is eyewear or headwear, attachment mechanism 16 may fit around a user's head, and when computing device 10 is a glove, attachment mechanism 16 may fit around a hand of a user.

UID 12 of computing device 10 may include a respective input and/or output device for computing device 10. UID 12 may be implemented using one or more various technologies. For instance, UID 12 may function as input device using a presence-sensitive input screen, such as a resistive touchscreen, a surface acoustic wave touchscreen, a capacitive touchscreen, a projective capacitance touchscreen, a pressure sensitive screen, an acoustic pulse recognition touchscreen, or another presence-sensitive display technology. UID 12 may function as output (e.g., display) device using any one or more display devices, such as a liquid crystal display (LCD), a dot matrix display, a light emitting diode (LED) display, an organic light-emitting diode (OLED) display, e-ink, or similar monochrome or color displays capable of outputting visible information to a user of computing device 10.

In some examples, UID 12 may include a presence-sensitive display that may include a display device and receive tactile input from a user of computing device 10. UID 12 may receive indications of tactile input by detecting one or more gestures from a user (e.g., the user touching or pointing to one or more locations of UID 12 with a finger or a stylus pen). UID 12 may present output to a user, for instance at a presence-sensitive display. UID 12 may present the output as a graphical user interface (e.g., a user interface for viewing an alert based on notification information), which may be associated with functionality provided by computing device 10. For example, UID 12 may present various user interfaces related to the functionality of computing platforms, operating systems, applications, and/or services executing at or accessible by computing device 10 (e.g., notification services, electronic message applications, Internet browser applications, mobile or desktop operating systems, etc.). A user may interact with a user interface presented at UID 12 to cause computing device 10 to perform operations relating to functions.

UI module 20 may receive and interpret inputs detected at UID 12 (e.g., as a user provides one or more gestures at one or more locations of UID 12 at which a user interface is displayed) and input detected at other input devices of computing device 10 (e.g., microphones, physical buttons, etc.). UI module 20 may relay information about the input detected at computing device 10 to one or more associated platforms, operating systems, applications, and/or services executing at computing device 10, to cause computing device 10 to perform functions.

UI module 20 also may receive information and instructions from one or more associated platforms, operating systems, applications, and/or services executing at computing device 10 (e.g., notification client modules 22, etc.) for generating a graphical user interface or for providing notification information, such as a representation of the notification (e.g., graphical representation) or indication of the notification (e.g., an alert regarding the presence of the notification). The alert may provide feedback in the form on visual, audible, or tactile feedback to the user associated with computing device 10. In addition, UI module 20 may act as a respective intermediary between the one or more associated platforms, operating systems, applications, and/or services executing at computing device 10 and various output devices of computing device 10 (e.g., UID 12, one or more sensors 14, a speaker, a LED indicator, other output devices, etc.) to produce output (e.g., a graphic, a flash of light, a sound, a tactile response such as a haptic response, etc.) with computing device 10.

One or more sensors 14 may include any type of sensor configured to sense a physiological signal or state of the user and generate a signal indicative of the sensed physiological parameter. For example, a GSR sensor may be housed on a skin-facing surface of housing 18 and/or attachment mechanism 16 and include two or more electrodes for detecting the resistance between the two electrodes. This resistance may change due to perspiration from the user during stressful or active situations of the user, and the GSR sensor may use this change in resistance to generate a corresponding signal indicative of the physiological condition. In other examples, housing 18 may contain a temperature sensor (e.g., thermistor or thermocouple) to sense the user's temperature, a pulse oximeter to sense oxygen saturation and/or pulse rate, one or more pressure sensors, or two or more electrodes for detecting an electrogram of the user. One or more sensor 14 may also include one or more gyroscopes, accelerometers, or proximity sensors. Any of these one or more sensors 14 may be used to generate the physiological parameter computing device 10 analyzes to determine which notification information to output for delivery to the user.

Notification client module 22 may perform functions associated with receiving, managing, outputting, and otherwise handling at least a portion of the notification information generated and/or received by platforms, applications, and services executing at computing device 10. Notification client module 22 may receive notification information from notification host module 62 of information server system 60 and output the received notification information to a recipient platform, application, and/or service executing at computing device 10. The received notification information from notification host module 62 may be in the form of a request to output and/or deliver the notification information to the user. Notification client module 22 may receive notification information generated by a platform, application, and/or service executing at computing device 10, and output the received notification information to information server system 60 over links 32. Notification client module 22 also may cause UI module 20 to output representations of the notifications (e.g., graphical representations of the notification) and/or output indications of the notification (e.g., one or more alerts) to indicate the receipt of notification information by computing device 10.

As used throughout the disclosure, the term “notification information” is used to describe various types of information that may indicate the occurrence of an event associated with various platforms, applications, and services executing within an execution environment at one or more computing devices, such as computing device 10. For example, notification information may include, but is not limited to, information regarding an event such as: the receipt of a communication message (e.g., e-mail, instant message, SMS, etc.) by a messaging account associated with a user of computing device 10, the receipt of information by a social networking account associated with a user of computing device 10, a reminder of a calendar event (meetings, appointments, etc.) associated with a calendar account of a user of computing device 10, information generated and/or received by a third-party application executing at computing device 10, the transmittal and/or receipt of inter-component communications between two or more components of platforms, applications, and/or services executing at computing device 10, etc.

Context module 24 may receive and aggregate physiological parameters received from the one or more sensors 14. Based on the context of the user, such as the one or more physiological parameters regarding the user, context module 24 may infer a physiological condition of the user at a particular (e.g., current) time. In some examples, context module 24 may utilize other factors, such as the time of day, day of the week, current calendar events, or any other information to refine the possible condition or conditions of the user. In this manner, context module 24 may output the physiological parameter or indication of the physiological parameter. In some examples, context module 24 may also predict, infer, or otherwise determine the condition of the user based on the received physiological parameters. Context module 24 may thus output information (e.g., physiological parameters and/or the determined physiological condition) to notification client module 22 that indicates whether the user is stressed, relaxed, or neither stressed nor relaxed at a current time, based on the information received by context module 24.

In some examples, context module 24 may determine whether or not the physiological parameters are received during a physical activity of the user or a non-physical activity of the user. When the user is physically active, one or more of the physiological parameters may indicate a stressful condition when the user is instead active and perspiring or having an elevated heart rate. Context module 24 may be configured to reduce these false positives by incorporating activity information to the received physiological parameters. For example, context module 24 may detect physical activity by analyzing accelerometer data and annotate physiological parameters with a flag that indicates the user was physically active when the physiological parameter was generated. Therefore, context module 24 may not determine that the user is stressed or excited in these situations or context module 24 may present the flag to notification client module 22 along with the physiological parameter for determination.

Using the information received by context module 24, notification client module 22 may be configured to determine whether to output some or all of the notification information received by notification host module 62. Notification client module 22 may be configured to compare one or more values of the physiological parameters or the physiological condition received from context module 24 to one or more thresholds or rules for the respective parameters or conditions. In some examples, a threshold may be applicable to any notification information such that notification client module 22 determines not to output all notification information in response to a value of a physiological condition exceeding the threshold. In other examples, a threshold may be applicable to a certain type or types of notification information (e.g., communication messages or calendar events) or notification information originating from certain contacts or other devices (e.g., a device or account associated with one or more other users). Notification client module 22 may utilize rules to determine whether or not to output notification information for complex situations that involve one threshold, multiple thresholds, or complex relationships between physiological parameter values or physiological conditions (e.g., various ranges relating to respective output instructions).

Although notification client module 22 is described as determining whether or not to output notification information based on a physiological parameter, one or more other modules or components of computing device 10 may perform this task. For example, context module 24 or UI module 20 may be configured to perform these determinations. In other examples, a separate determination module may be configured to determine whether or not notification information should be output for delivery to the user. Alternatively, computing device 10 may transmit the physiological parameters from the one or more sensors 14 and/or information generated by context module 24 to information server system 60, and information server system 60 may determine whether or not to output the notification information.

Although a physiological parameter is referenced herein, the physiological parameter may refer to any information representing a certain physiological signal or state. In this manner, a physiological parameter may be binary such as a sub-threshold or supra-threshold physiological event. Alternatively, the physiological parameter may have a variety of different values that represent the variations of the parameter. For example, a GSR physiological parameter may be represented by a certain impedance or conductance value for the user. In this manner, a physiological parameter may be a variable that computing device 10 uses to determine whether or not to output at least some notification information.

In some examples, context module 24 may obtain physiological parameters and/or other information related to a user of computing device 10 in substantially real-time (e.g., as fast as the one or more sensors 14 can generate and transmit the information). In some examples, context module 24 may obtain physiological parameters and other information related to the user of computing device 10 periodically (e.g., repeatedly with a defined period) or aperiodically (e.g., responsive to computing device 10 receiving notification information from information server system 60). In some examples, context module 24 may receive information related to a user of computing device 10 at various earlier times and use the earlier information to learn and/or produce rules for discerning whether a user of computing device 10 is stressed, relaxed, and/or neither stressed nor relaxed, at a later time.

Computing device 10 and information server system 60 may only collect or make use of information associated with a user of computing device 10 (e.g., notification information, contextual information, etc.) after providing the user with an opportunity to provide input at computing device 10 to control whether programs or features of information server system 60 and computing device 10 can collect and make use of user information (e.g., information about a user's current location, physiological condition, etc.), or to dictate whether and/or how information server system 60 and computing device 10 may receive content that may be relevant to the user. In addition, certain data may be treated in one or more ways before it is stored or used by information server system 60 and computing device 10, so that personally-identifiable information is removed.

In operation, information server system 60 may receive notification information associated with a user of computing device 10, e.g., from a content server associated with one or more accounts associated with the user of computing device 10. Notification host module 62 may output the notification information over network link 32N to network 30. Notification client module 22 of computing device 10 may receive the notification information from network 30 via network link 32A.

Although system 1 is described as an example push notification system (e.g., a cloud-based notification system), the components of system 10 may additionally or alternatively operate as a pull notification system in which the end-user device (e.g., computing device 10) executes notification client module 22 to request or pull notification information from information server system 60. In other examples, computing device 10 may execute one or more applications (e.g., applications resident on computing device 10) that generate notification information for presentation to the user of computing device 10. In this manner, some or all notification information may be defined by an application and/or operating system executing on computing device 10, instead of the notification information being received from a remote device such as information server system 60. The notification information may be generated based on information received from a remote device (e.g., information server system 60) or information generated by the same or different application or operating system executing on computing device 10. In addition, system 10 may implement several different modes of generating notification information. For example, system 10 may implement push and/or pull notification systems that include information service system 60 while also including notification information independently generated by computing device 10 (e.g., one or more applications or the operating system of computing device 10 that generate notification information).

Notification information described herein may indicate, or notify, a user of any type of event. For example, notification information may be generated, responsive to receiving and to represent, a received communication (e.g., a text message, an email, an instant message, a voicemail, a phone call, etc.), a calendar event, an application status, a system condition, or any other event. An application status may be a status of an application or operating system executing on computing device 10. A system condition may include, for example, the availability of a networked service, a network connection supported by computing device 10, a power supply status for computing device 10, a software update availability for computing device 10, or any other such condition. In this manner, notification information may be generated to indicate any event or condition for which a user may be notified. Notification information may or may not require user input in response to the notification information.

Throughout the disclosure, examples are described where a computing device and/or a computing system may analyze information (e.g., physiological parameters, notification information, etc.) associated with a computing device only if the computing device receives permission from the user to analyze the information. For example, in situations discussed below in which the computing device may collect or may make use of information associated with the user, the user may be provided with an opportunity to provide input to control whether programs or features of the computing device can collect and make use of user information (e.g., information about a user's current physiological condition, current location, etc.), or to dictate whether and/or how to the computing device may receive content that may be relevant to the user. In addition, certain data may be treated in one or more ways before it is stored or used by the computing device and/or computing system, so that personally-identifiable information is removed. For example, a user's identity may be treated so that no personally identifiable information can be determined about the user. Thus, the user may have control over how information is collected about the user and used by the computing device.

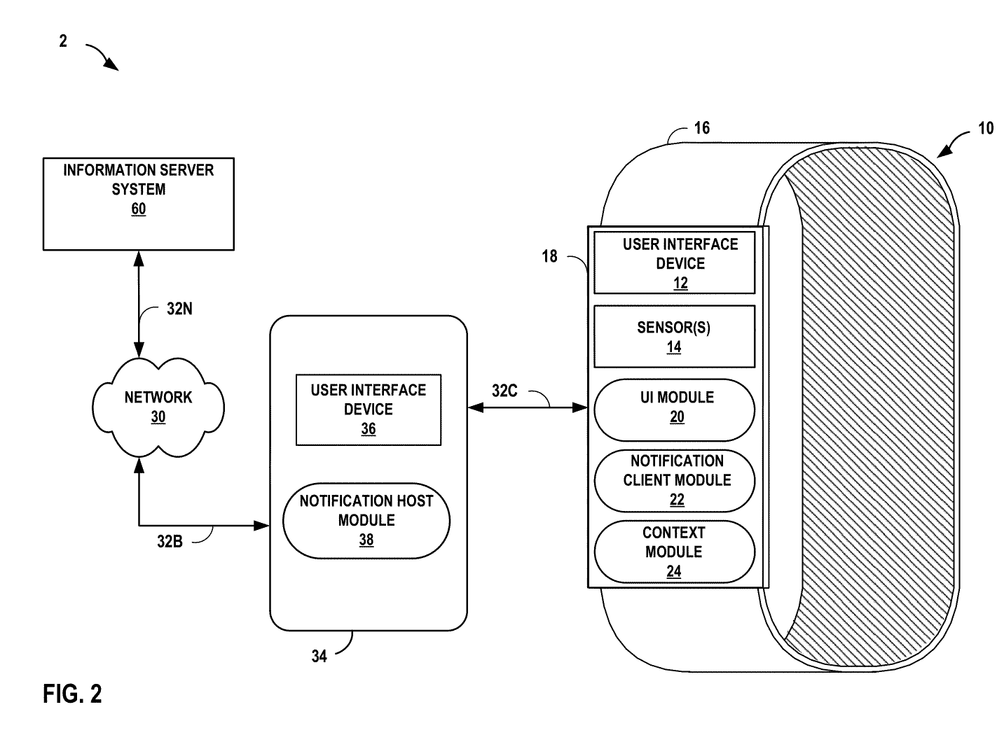

FIG. 2 is a conceptual diagram illustrating an example computing system 2 that includes mobile computing device 34 that outputs notification information to an example computing device 10, in accordance with one or more aspects of the present disclosure. The example system 2 is substantially similar to system 1 of FIG. 1. However, mobile computing device 34 may include notification host module 36 that is substantially similar to notification host module 62 of FIG. 1. Computing system 2 of FIG. 2 is an example computing system that includes computing device 10, mobile computing device 34, information server system 60, and network 30, that may perform various tasks described herein.

Mobile computing device 34 and information server system 60 may send and receive data across network 30 using any suitable communication techniques known in the art. Mobile computing device 34 may be operatively coupled to network 30 using network link 32B (similar to network link 32A of FIG. 1) and information server system 60 may be operatively coupled to network 30 by network link 32N. Computing device 10 may communicate with mobile computing device 34 via link 32C. Network links 32B, 32C, and 32N may utilize the same or different communication protocols. Network link 32C may utilize device-to-device communication protocol such as a Bluetooth® protocol or other wireless or wired communication link.

Information server system 60 may distribute various information to mobile computing device 34. Mobile computing device 34 may be a smartphone, tablet computing device, notebook computer, or any other computing device to which computing device 10 can communication. Mobile computing device 34 may include a user interface device 36 that may include similar components to that of user interface device 12 of computing device 10. As shown in FIG. 2, computing device 10 may be configured to communicate directly with mobile computing device 34 and/or server system 60. However, computing device 10 may be configured to operate cooperatively with mobile computing device 34. For example, computing device 10 may operate as a slave device and mobile computing device 34 may operate as a master device that transmits requests or commands to computing device 10. In practice, a user may carry mobile computing device 34 and wear computing device 10, such that mobile computing device 34 and computing device 10 are in communication via link 32C. Computing device 10 may operate as a user interface extension to user interface device 36, for example. In other examples, computing device 10 may additionally, or alternatively, communicate with other devices (e.g., information server system 60) via a link to network 30.

With regard to notification information, notification host module 38 may transmit requests for outputting notification information to computing device 10. In other words, mobile computing device 34 may receive a communication message or other event, and notification host module 38 may generate a request to output notification information related to the event and transmit the request to computing device 10 via link 32C. Similar to the description provided in FIG. 1, computing device 10 may then determine whether or not to output the notification information based on one or more physiological parameters.

In other examples, mobile computing device 34 may include context module 24 and/or notification client module 22. Mobile computing device 34 may receive notification information from information server system 60 and physiological parameters from computing device 10. Mobile computing device 34 may then, utilizing a notification client module, determine, based on the received physiological parameters, whether or not to output some or all of the notification information. In some examples, mobile computing device 34 may selectively output the notification information via user interface device 36. Mobile computing device 34 may alternatively, or additionally, selectively output notification information for transmission to computing device 10, and user interface device 12 may present notification information that has not been withheld by mobile computing device 34. In some examples, representations of the notification may be presented by mobile computing device 34 whereas indications of the notification (e.g., alerts) may be presented by computing device 10 and/or mobile computing device 34. In any example, notification information that has been selectively output may be delivered to the user by one or more components of mobile computing device 34 and/or computing device 10.

Although a single computing device 10 and single mobile computing device 34 are described in FIG. 2, any number of computing devices 10 and mobile computing devices 34 may communicate with each other and cooperatively determine whether or not to output notification information to the user or otherwise control the delivery of information to the user.

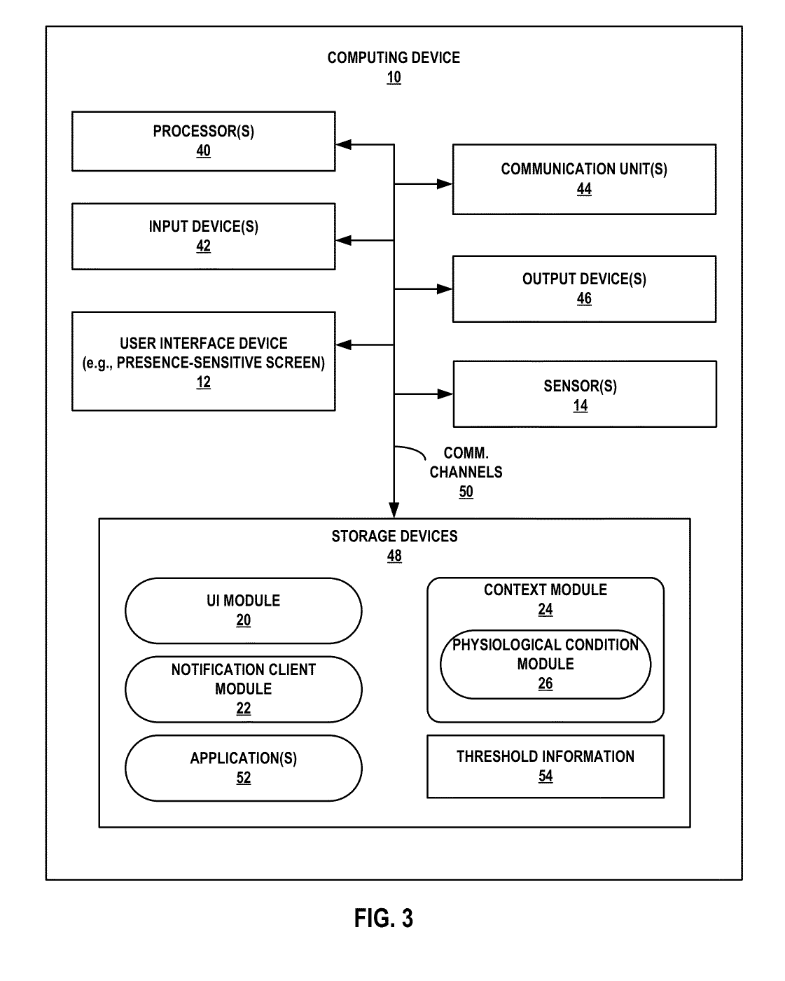

FIG. 3 is a block diagram illustrating an example computing device 10 configured to manage notification information based on one or more physiological parameters. Computing device 10 of FIG. 3 is described below within the context of FIGS. 1 and 2. FIG. 3 illustrates only one particular example of computing device 10, and many other examples of computing device 10 may be used in other instances and may include a subset of the components included in example computing device 10 or may include additional components not shown in FIG. 3.

As shown in the example of FIG. 3, computing device 10 includes UID 12, one or more sensors 14, one or more processors 40, one or more input devices 42, one or more communication units 44, one or more output devices 46, and one or more storage devices 48. In the illustrated example, storage devices 48 of computing device 10 also include UI module 20, notification client module 22, context module 24, one or more applications 52, and threshold information 54. Context module 24 includes physiological condition module 26. Communication channels 50 may interconnect each of the components 12, 14, 20, 22, 24, 26, 28, 40, 42, 44, 46, 52, and 54 for inter-component communications (physically, communicatively, and/or operatively). In some examples, communication channels 50 may include a system bus, a network connection, an inter-process communication data structure, or any other method for communicating data. In examples in which any of modules 20, 22, 24, 26, 52, and 54 are provided as hardware, computing device 10 may include such hardware outside of any storage device 48.

One or more input devices 42 of computing device 10 may receive input. Examples of input are tactile, audio, video, or user input. Input devices 42 of computing device 10, in some examples, include a presence-sensitive input device (e.g., a touch sensitive screen, a presence-sensitive display), mouse, keyboard, voice responsive system, video camera, microphone, or any other type of device for detecting input from a human or machine.

One or more sensors 14 may include one or more physiological sensors for obtaining physiological parameter information associated with a user of computing device 10. For example, one or more sensors 14 may include a heart monitor sensor, a temperature sensor, a galvanic skin response sensor, an accelerometer, a gyroscope, a pressure sensor, a blood pressure sensor, and/or any other sensor for measuring a physiological parameter that computing device 10 may use for determining a physiological condition of a user. In some examples, input devices 42 may include one or more of sensors 14.

One or more output devices 46 of computing device 10 may generate output. Examples of output are tactile, audio, and video output. Output devices 46 of computing device 10, in some examples, include a presence-sensitive display, sound card, video graphics adapter card, speaker, cathode ray tube (CRT) monitor, liquid crystal display (LCD), a tactile output device (e.g., a haptic device) or any other type of device for generating output to a human or machine. One or more output devices 46 may deliver the representation of a notification and/or an indication of the notification to the user.

One or more communication units 44 of computing device 10 may communicate with external devices via one or more networks by transmitting and/or receiving network signals on the one or more networks. For example, computing device 10 may use communication unit 44 to transmit and/or receive radio signals on a radio network such as a cellular radio network. Likewise, communication units 44 may transmit and/or receive satellite signals on a satellite network such as a GPS network. Examples of communication unit 44 include a network interface card (e.g. such as an Ethernet card), an optical transceiver, a radio frequency transceiver, a GPS receiver, or any other type of device that can send and/or receive information. Other examples of communication units 44 may include Bluetooth®, GPS, 3G, 4G, and Wi-Fi® radios found in mobile devices as well as Universal Serial Bus (USB) controllers.

In some examples, UID 12 of computing device 10 may include functionality of input devices 42 and/or output devices 46. In the example of FIG. 2, UID 12 may be or may include a presence-sensitive input device. In some examples, a presence-sensitive input device may detect an object at and/or near the presence-sensitive input device. As one example range, a presence-sensitive input device may detect an object, such as a finger or stylus that is within two inches or less of the presence-sensitive input device. In another example range, a presence-sensitive input device may detect an object six inches or less from the presence-sensitive input device, and other ranges are also possible. The presence-sensitive input device may determine a location (e.g., an (x,y) coordinate) of the presence-sensitive input device at which the object was detected. The presence-sensitive input device may determine the location selected by the input device using capacitive, inductive, and/or optical recognition techniques. In some examples, presence-sensitive input device provides output to a user using tactile, audio, or video stimuli as described with respect to output device 46, and may be referred to as a presence-sensitive display.

While illustrated as an internal component of computing device 10, UID 12 also represents an external component that shares a data path with computing device 10 for transmitting and/or receiving input and output. For instance, in one example, UID 12 represents a built-in component of computing device 10 located within and physically connected to the external packaging of computing device 10 (e.g., a screen on a mobile phone or wearable computing device). In another example, UID 12 represents an external component of computing device 10 located outside and physically separated from the packaging of computing device 10 (e.g., a monitor, a projector, etc. that shares a wired and/or wireless data path with computing device 10).

One or more storage devices 48 within computing device 10 may store information for processing during operation of computing device 10. In some examples, storage device 48 is a temporary memory, meaning that a primary purpose of storage device 48 is not long-term storage. Storage devices 48 on computing device 10 may configured for short-term storage of information as volatile memory and therefore not retain stored contents if powered off. Examples of volatile memories include random access memories (RAM), dynamic random access memories (DRAM), static random access memories (SRAM), and other forms of volatile memories known in the art.

Storage devices 48, in some examples, also include one or more computer-readable storage media. Storage devices 48 may be configured to store larger amounts of information than a temporary memory. Storage devices 48 may further be configured for long-term storage of information as non-volatile memory space and retain information after power on/off cycles. Examples of non-volatile memories include magnetic hard discs, optical discs, floppy discs, flash memories, or forms of electrically programmable memories (EPROM) or electrically erasable and programmable (EEPROM) memories. Storage devices 48 may store program instructions and/or data associated with UI module 20, notification client module 22, context module 24, physiological condition module 26, one or more applications 52, and threshold information 54.

One or more processors 40 may implement functionality and/or execute instructions within computing device 10. For example, processors 40 on computing device 10 may receive and execute instructions stored by storage devices 48 that execute the functionality of UI module 20, notification client module 22, context module 24, physiological condition module 26, and/or one or more applications 52. These instructions executed by processors 40 may cause computing device 10 to store information within storage devices 48 during program execution. Processors 40 may execute instructions of modules 20-26 and 52 to cause notification client module 22 to determine whether or not to output, for delivery to the user, notification information and selectively output the notification information based on the determination. That is, modules 20-28 and 52 may be operable by processors 40 to perform various actions, including determining whether to output notification information (e.g., determining whether to withhold notification information or output the notification information delivery to the user).

In accordance with aspects of this disclosure, context module 24 of computing device 10 may receive physiological parameters (e.g., values of respective physiological parameters) from one or more sensors 14 that are representative of a physiological condition of the user associated with computing device 10. In some examples, context module 24 may pass the physiological parameter to notification client module 22 for determination of whether or not some or all of received notification information should be output for delivery to the user. In other examples, context module 24 may perform one or more operations on the received physiological parameters before sending related physiological information to notification module 22. Context module 24 may sample a subset of physiological parameters and send the subset of parameters to notification client module 22, calibrate the physiological parameters to a certain unit corresponding to the thresholds or rules stored as threshold information 54, or otherwise process the physiological parameters for use in controlling the flow of information within or from computing device 10.

In other examples, context module 24 may analyze the physiological parameters to determine the physiological condition experienced by the user. Physiological condition module 26 may be configured to compare the received values of the respective one or more physiological parameters to one or more thresholds or rules to select at least one corresponding physiological condition. Context module 24 may then transmit the selected physiological condition to notification client module 22 for determination of whether or not notification information should be withheld from being output for delivery to the user. In some examples, physiological condition module 26 and/or context module 24 may be configured to differentiate between physiological parameters due to physical activity of the user or non-activity related physiological conditions. Since physical activity (e.g., exercise such as hiking, running, or riding a bicycle) may cause one or more physiological parameters of the user to mimic another physiological condition, computing device 10 may use movement information received from one or more accelerometers, gyroscopes, or other such sensors 14 to differentiate the physiological conditions from physical activity. In some examples, computing device 10 may also control information flow, such as determine which notification information to output, according to the physical activity detected of the user.

Notification client module 22 may be configured to determine, based on one or more physiological parameters, whether to output notification information received from a notification host module such and selectively output, based on the determination, the notification information. In this manner, notification client module 22 may determine whether to withhold certain notification information or allow the notification information to be output for delivery to the user. Although notification client module 22 may determine whether or not to output notification information from one physiological parameter (e.g., one or more values of the physiological parameter), notification client module 22 may determine whether or not to output notification information based on two or more different physiological parameters. For example, notification client module 22 may be configured to receive indications of a first and a second physiological parameter each representative of the physiological condition of the user, determine, based on a first physiological parameter and a second physiological parameter, whether to output the notification information. For example, notification client module 22 may utilize physiological parameters from a GSR sensor and a pulse rate sensor. In other examples, more than two different physiological parameters may be used to control the flow of notification information.

Notification client module 22 may determine whether to output notification information by comparison of physiological parameters to respective thresholds or rules stored in threshold information 54. For example, notification client module 22 may be configured to compare a value of the physiological parameter to a threshold value, determine that the value exceeds the threshold value, and, responsive to determining that the value exceeds the threshold value, withhold output of at least some notification information. Threshold information 54 may store one or more thresholds, rules, or any other guidelines that allow notification client module 22 to determine when to withhold or allow notification. In some examples, the thresholds or rules may apply to any and all notification information received by computing device 10. In other words, notification client module 22 may withhold all notification information when one or more physiological parameters instruct notification client module 22 to do so. Alternatively, each of the thresholds or rules may be applicable to a respective type of notification information (e.g., the representation of the notification or one or more indications of the notification), a source of the notification information (e.g., a particular application or service such as an email message service, text message service, or social media message service), and/or another user associated with the notification information. In this manner, notification client module 22 may be configured to selectively withhold a first subset of the received notification information and output a second subset of the received notification information based on one or more received physiological parameters.

In addition, notification client module 22 may be configured to determine whether one or both of a representation of the notification and an indication of the notification should be withheld based on the physiological parameter. The notification information for a single event may include both a representation of the notification (e.g., a graphical representation of the notification regarding the event such as a portion of an email message) and an indication of the notification (e.g., at least one of a visual alert for the notification, an audible alert for the notification, and a tactile alert for the notification where each alert may indicate the presence of the representation of the notification). Notification client module 22 may this be configured to withhold some of the notification information while outputting other notification information. For example, notification client module 22 may, based on one or more physiological parameters, output the representation of the notification and withhold the indication of the notification. The representation of the notification may be delivered via a display device that is only visable when the user turns on the display device, so the representation of the notification may be minimally intrusive. However, the indication of the notification may be an audible sound, tactile feedback, activated light, or any other type of alert that may disturb the user when the physiological parameter suggests the user is not to be disturbed due to a higher level of stress or concentration, for example.

As indicated above, notification client module 22 may withhold some or all of the notification information requested by a notification host module to be delivered to the user. However, notification client module 22 may also hold the withheld notification information for possible output at a later time. Notification client module 22 may continually or periodically monitor new values of the one or more physiological parameters and compare the new values to the respective thresholds or rules. Therefore, notification client module 22 may, subsequent to withholding output of notification information, receive an indication of a second value of the physiological parameter, determine that the second value is below a respective threshold value, and responsive to determining that the second value is below the threshold value, output the notification information. In this manner, notification client module 22 may be configured to temporarily withhold notification information. Alternatively, notification client module 22 may be configured to permanently withhold notification information by not outputting the notification information once it has been withhold.

In some examples, computing device 10 may continue to output other information for delivery to the user even when some notification information is withheld. For example, UI module 20 may control user interface device 12 to output a graphical user interface having information for view by the user, and withheld notification information may merely not be output for delivery to the user. In other words, the graphical user interface generated by computing device 10 would be without the notification information. Alternatively, determination by notification client module 22 to not output notification information may also include a command that instructs computing device 10 (e.g., a wearable computing device) to turn off a display device (e.g., user interface device 12) and/or other output devices 46. Typically, this turning off of a display device may be reserved for instances in which notification client module 22 determines that all notification information should be withheld. Therefore, the determination to not output notification information may represent a type of “do not disturb” signal that causes computing device 10 to turn off one or more output devices that interface with the user. Turning off the display device may also include maintaining the display device in the off state if the display device is already turned off.

As described herein, the notification information may be associated with an event from a communication service, a calendar, an executing application, or other such sources. The notification information is unrelated to the one or more physiological parameters detected by the one or more sensors 14. In other words, the notification information does not provide an update or information describing the physiological parameter of the user or the user's physiological condition. Instead, the notification information that may be withheld may be related to events, applications, services, system conditions, or devices other than the physiological parameters or physiological conditions detected by the one or more sensors 14. For example, the notification information may be indicative of an event related to at least one application executing on computing device 10, such as an instant message application, an email application, a text message application, a calendar application, system condition such as a battery status application, or any other such applications. Notification client module 22 may be configured to support push and/or pull notification systems and/or support notification information generated by an application or the operating system of computing device 10. In some examples, notification client module 22 may generate notification information at the request of, or in response to receiving information from, the operating system or another application executing on computing device 10.

Although computing device 10 is described in relation to determining whether to output notification information based on the physiological parameters, computing device 10 may be configured to manage other information flow. For example, computing device 10 may include a module configured to filter a plurality of items (e.g., search results, available executable applications, contacts, or any other information) based on one or more physiological parameters and output the subset of filtered. In this manner, computing device 10 may be configured to limit the plurality of items based on the condition of the user. For example, computing device 10 may, based on one or more physiological parameters, remove items less relevant to the condition of the user and/or reduce the number of items for presentation to the user.

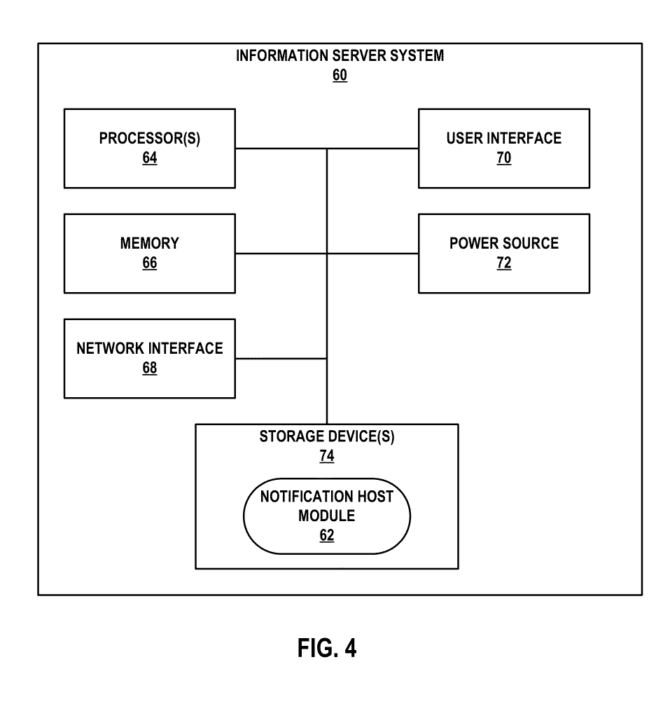

FIG. 4 is a block diagram illustrating an example information server system 60 of FIG. 1 configured to output notification information. FIG. 4 illustrates only one particular example of information server system 60 (e.g., one or more computing devices), and many other example embodiments of information server system 60 may be used in other instances. For example, information server system 60 may include additional components, run multiple different applications, and/or include multiple different servers. In some examples, information server system 60 may include multiple servers in communication over network 30 and/or another network. As shown in the specific example of FIG. 4, information server system 60 includes one or more processors 64, memory 66, a network interface 68, one or more storage devices 74, user interface 70, power source 72, and notification host module 62. Server system 60 may also include an operating system that includes modules and/or applications that are executable by processors 64. Each of components 64, 66, 68, 70, 72, 74, and 62 may be interconnected (physically, communicatively, and/or operatively) for inter-component communications. Server system 60 may also be in communication with a repository or even include a repository as one of storage devices 74.

Processors 64, in one example, are configured to implement functionality and/or process instructions for execution within server system 60. For example, processors 64 may be capable of processing instructions stored in memory 66, instructions stored on storage devices 74, or instructions stored in a repository. Notification host module 62 may be a hardware and/or software module configured to perform the various functions described herein related to selecting or determining which location indicators from multiple devices are representative of the location of a user associated with the computing devices. Notification host module 62 may, for example, generate notification information such as requests to output notification information based on one or more events that occur. The requests to output notification information may be received from one or more other applications, devices, and/or systems. Notification host module 62 may be configured to support (and be a component of) push and/or pull notification systems described herein for transmitting notification information to devices such as computing device 10. In other examples, processors 64 may at perform some or all of the functions of notification host module 62. Alternatively, server system 60 may offload some or all of the functions of notification host module 62 to computing device 10, computing device 32, other network servers, or other computing devices.

Memory 66, in one example, is configured to store information within server system 60 during operation. Memory 66, in some examples, is described as a computer-readable storage medium. In some examples, memory 66 is a temporary memory, meaning that a primary purpose of memory 66 is not long-term storage. Memory 66, in some examples, is described as a volatile memory, meaning that memory 66 does not maintain stored contents when the computer is turned off. Examples of volatile memories include random access memories (RAM), dynamic random access memories (DRAM), static random access memories (SRAM), and other forms of volatile memories known in the art. In some examples, memory 66 is used to store program instructions for execution by processors 64.

Storage devices 74, in some examples, also include one or more computer-readable storage media. Storage devices 74 may be configured to store larger amounts of information than memory 74. Storage devices 74 may further be configured for long-term storage of information. In some examples, storage devices 74 include non-volatile storage elements. Examples of such non-volatile storage elements include magnetic hard discs, optical discs, floppy discs, flash memories, or forms of electrically programmable memories (EPROM) or electrically erasable and programmable (EEPROM) memories. Repository 24 may also include one or more computer-readable storage media, in some examples.

Server system 60, in some examples, also includes a network interface 68 configured to communicate with other devices (e.g., computing device 10 and/or computing device 32) and transmit and/or receive data via network 30, for example. Network interface 68 may be a network interface card, such as an Ethernet card, an optical transceiver, a radio frequency transceiver, or any other type of device that can send and receive information. In alternative examples, network interface 68 may include Bluetooth, 3G, 4G, and WiFi radios in mobile computing devices as well as USB.

User interface 70 may be configured to receive input from a user (e.g., tactile, audio, or video feedback) when direct interaction with server system 60 is desired. User interface 70 may include a touch-sensitive and/or a presence-sensitive screen or display, mouse, a keyboard, a voice responsive system, or any other type of device for detecting a command from a user. User interface 70 may also include, combined or separate from input devices, output devices. User interface 70 may include a speaker, a liquid crystal display (LCD), light emitting diode (LED) array, or any other type of device that can generate intelligible output to a user. Power source may include a battery and/or circuit for generating power from an AC or DC power source.

In other examples, server system 60 may include additional components to perform functionality described herein. Any applications or modules (e.g., notification host module 62) implemented within or executed by information server system 60 may be implemented or contained within, operable by, executed by, and/or be operatively/communicatively coupled to components of information server system 60 (e.g., processors 64, memory 66, network interface 68, and/or storage devices 74).

As described herein, notification host module 62 may generate notification information for transmission to computing device 10 or any other notification client module. In some examples, notification host module 62, or another module of server system 60, may be configured to receive, from a wearable computing device (e.g., computing device 10) and via network 30, the indication of the physiological parameter. Notification host module 62 may then determine, based on the physiological parameter, whether or not some or all of the notification information should be output for delivery to the user. Notification host module 62 may then only output the subset of notification information that is not withheld due to the physiological parameter or condition of the user. In some examples, where server system 60 may provide greater processing capability, distribution of physiological parameter analysis and determination of information flow away from computing device 10 (e.g., a wearable computing device) may reduce processing times and conserve power of computing device 10.

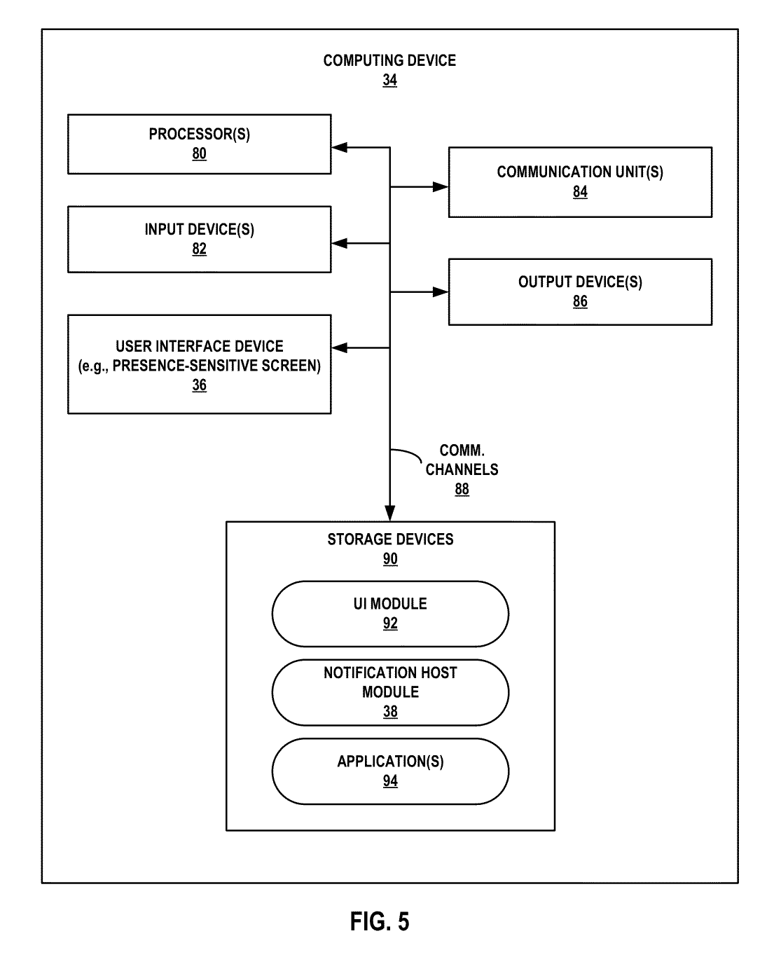

FIG. 5 is a block diagram illustrating an example mobile computing device 34 of FIG. 2 configured to output notification information. Computing device 34 may be a mobile computing device and configured to communicate with computing device 10 of FIG. 2. As shown in FIG. 5, computing device 34 includes UID 36, one or more processors 80, one or more input devices 82, one or more communication units 84, one or more output devices 86, one or more communication channels 88, and one or more storage devices 90. These components of computing device 34 may be similar, in form and/or function, to the respective components described herein with respect to computing device 10 (e.g., UID 12, one or more processors 40, one or more input devices 42, one or more communication units 44, one or more output devices 46, one or more communication channels 50, and one or more storage devices 48).

In the illustrated example, storage devices 90 of computing device 34 also include UI module 92, notification host module 38, and, one or more applications 94. Communication channels 88 may interconnect each of the components 80, 82, 36, 84, 86, 90, 92, 38, and 94 for inter-component communications (physically, communicatively, and/or operatively). In some examples, communication channels 50 may include a system bus, a network connection, an inter-process communication data structure, or any other method for communicating data. In examples in which any of modules 92, 38, and 94 are provided as hardware, computing device 34 may include such hardware outside of any storage device 90.

One or more communication units 84 of computing device 34 may communicate with external devices via one or more networks by transmitting and/or receiving network signals on the one or more networks and or other devices (e.g., computing device 10) directly via direct device-to-device communication. For example, computing device 34 may use communication unit 84 to transmit and/or receive radio signals on a radio network such as a cellular radio network. Likewise, communication units 84 may transmit and/or receive satellite signals on a satellite network such as a GPS network. Examples of communication unit 84 include a network interface card (e.g. such as an Ethernet card), an optical transceiver, a radio frequency transceiver, a GPS receiver, or any other type of device that can send and/or receive information. Other examples of communication units 84 may include Bluetooth®, GPS, 3G, 4G, and Wi-Fi® radios found in mobile devices as well as Universal Serial Bus (USB) controllers.

One or more processors 80 may implement functionality and/or execute instructions within computing device 34. For example, processors 80 on computing device 34 may receive and execute instructions stored by storage devices 90 that execute the functionality of UI module 92, notification host module 38, and/or one or more applications 94. These instructions executed by processors 80 may cause computing device 34 to store information within storage devices 90 during program execution. Processors 80 may execute instructions of modules 92, 38, and or 94 to generate notification information, pass along notification information to computing device 10, determine whether to withhold some or all notification information, or otherwise perform processes described herein. For example, processors 80 may execute notification host module 38 to perform the steps described herein with respect to notification host module 62 of information system server 60. In this manner, notification host module 62 may generate notification information and requests to output the notification information and transmit the requests to computing device 10.

However, notification host module 38, processors 80, and any other module of computing device 34 may be configured to perform some or all of the tasks described with respect to computing device 10 (e.g., a wearable computing device). For example, computing device 34 may receive physiological parameters, determine physiological conditions based on the parameters, and/or determine whether or not to output some or all of the notification information based on the physiological parameters and/or conditions. In this manner, computing device 34 and computing device 10 may operate in communication as a distributed system to achieve the functions and perform the various processes described herein.

Notification host module 38 may be configured to generate notification information as a part of a push and/or pull notification system, such as described with respect to notification host module 62 of FIG. 4. In addition, or alternatively, notification host module 38 may support push and/or pull notification systems and/or support notification information generated by an application or operating system executing on computing device 34. In some examples, notification host module 38 may generate notification information at the request of, or in response to receiving information from, the operating system or another application executing on computing device 34. Computing device 34 may include separate modules for supporting respective push notification systems, pull notification systems, and/or locally generated notification information.

In some examples, various thresholds, rules, or any other instructions that govern which physiological parameters are used to control information flow and the values of each parameter required to alter information flow may be predetermined. For example, these thresholds or rules may be default instructions stored by a manufacturer of computing device 10 or generated by one or more services associated with the respective notification information. In other examples, computing device 10, computing device 34, or any other computing device may be configured to receive user input specifying one or more aspects related to the flow of information, such as when to withhold notification information or how to filter items based on physiological parameters. User input may specify the value of one or more thresholds or variables of one or more rules that control when certain information is withheld, filtered, or otherwise altered. In addition, the user input may specify which types of notification information, which information sources (e.g., services or applications) can have information controlled by the physiological parameters, or even identify one or more other users from which information can be controlled based on the physiological parameters. In this manner, computing device 10 and/or computing device 34 may be configured to allow some or full control over the management of information flow based on physiological parameters.

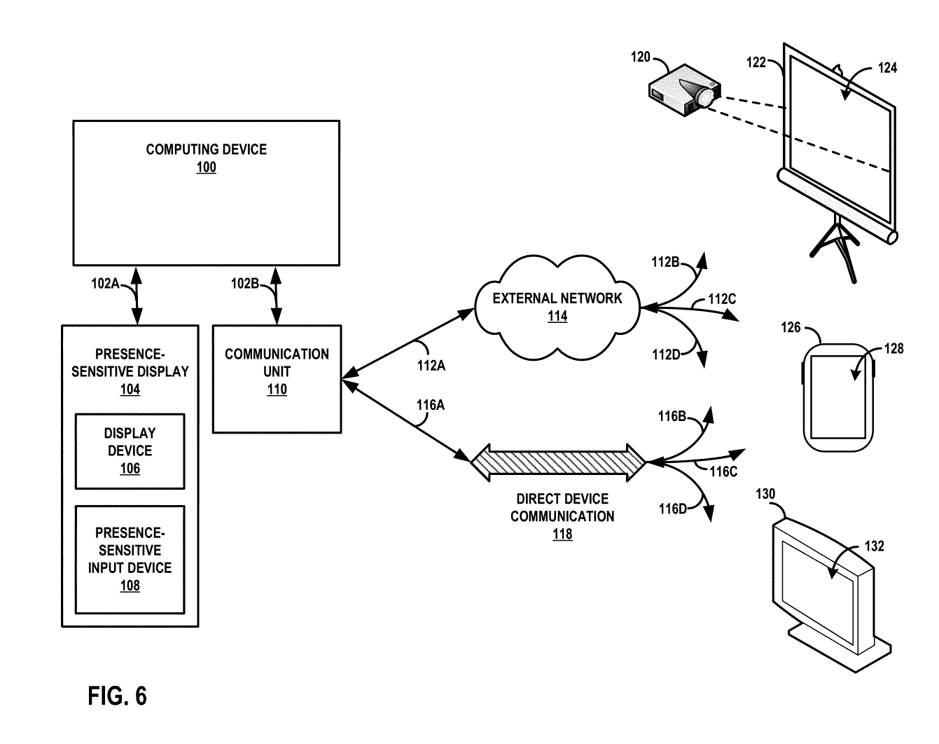

FIG. 6 is a block diagram illustrating an example computing device 100 that outputs graphical content for display at a remote device, in accordance with one or more techniques of the present disclosure. Graphical content, generally, may include any visual information that may be output for display, such as text, images, a group of moving images, etc. The example shown in FIG. 6 includes a computing device 100, presence-sensitive display 104, communication unit 110, projector 120, projector screen 122, mobile device 126, and visual display device 130. Although shown for purposes of example in FIGS. 1 and 2 as a stand-alone computing device 10 or mobile computing device 34, a computing device such as computing device 100 may, generally, be any component or system that includes a processor or other suitable computing environment for executing software instructions and, for example, need not include a presence-sensitive display.

As shown in the example of FIG. 6, computing device 100 may be a processor that includes functionality as described with respect to processor 40 in FIG. 3 or processor 80 in FIG. 5. In such examples, computing device 100 may be operatively coupled to presence-sensitive display 104 by a communication channel 102A, which may be a system bus or other suitable connection. Computing device 100 may also be operatively coupled to communication unit 110, further described below, by a communication channel 102B, which may also be a system bus or other suitable connection. Although shown separately as an example in FIG. 6, computing device 100 may be operatively coupled to presence-sensitive display 104 and communication unit 110 by any number of one or more communication channels.

In other examples, such as illustrated previously by computing devices 10 or 34 in FIGS. 1-2, a computing device may refer to a portable or mobile device such as mobile phones (including smart phones), laptop computers, smartwatch, etc. In some examples, a computing device may be a desktop computers, tablet computers, smart television platforms, cameras, personal digital assistants (PDAs), servers, mainframes, etc.

Presence-sensitive display 104, like user interface device 12 as shown in FIG. 1, may include display device 106 and presence-sensitive input device 108. Display device 106 may, for example, receive data from computing device 100 and display the graphical content. In some examples, presence-sensitive input device 108 may determine one or more user inputs (e.g., continuous gestures, multi-touch gestures, single-touch gestures, etc.) at presence-sensitive display 106 using capacitive, inductive, and/or optical recognition techniques and send indications of such user input to computing device 100 using communication channel 102A. In some examples, presence-sensitive input device 108 may be physically positioned on top of display device 106 such that, when a user positions an input unit over a graphical element displayed by display device 106, the location at which presence-sensitive input device 108 corresponds to the location of display device 106 at which the graphical element is displayed. In other examples, presence-sensitive input device 108 may be positioned physically apart from display device 106, and locations of presence-sensitive input device 108 may correspond to locations of display device 106, such that input can be made at presence-sensitive input device 108 for interacting with graphical elements displayed at corresponding locations of display device 106.

As shown in FIG. 6, computing device 100 may also include and/or be operatively coupled with communication unit 110. Communication unit 110 may include functionality of communication unit 44 as described in FIG. 2. Examples of communication unit 110 may include a network interface card, an Ethernet card, an optical transceiver, a radio frequency transceiver, or any other type of device that can send and receive information. Other examples of such communication units may include Bluetooth, 3G, and WiFi radios, Universal Serial Bus (USB) interfaces, etc. Computing device 100 may also include and/or be operatively coupled with one or more other devices, e.g., input devices, output devices, memory, storage devices, etc. that are not shown in FIG. 6 for purposes of brevity and illustration.

FIG. 6 also illustrates a projector 120 and projector screen 122. Other such examples of projection devices may include electronic whiteboards, holographic display devices, and any other suitable devices for displaying graphical content. Projector 120 and projector screen 122 may include one or more communication units that enable the respective devices to communicate with computing device 100. In some examples, the one or more communication units may enable communication between projector 120 and projector screen 122. Projector 120 may receive data from computing device 100 that includes graphical content. Projector 120, in response to receiving the data, may project the graphical content onto projector screen 122. In some examples, projector 120 may determine one or more user inputs (e.g., continuous gestures, multi-touch gestures, single-touch gestures, etc.) at projector screen using optical recognition or other suitable techniques and send indications of such user input using one or more communication units to computing device 100. In such examples, projector screen 122 may be unnecessary, and projector 120 may project graphical content on any suitable medium and detect one or more user inputs using optical recognition or other such suitable techniques.

Projector screen 122, in some examples, may include a presence-sensitive display 84. Presence-sensitive display 124 may include a subset of functionality or all of the functionality of UI device 12 as described in this disclosure. In some examples, presence-sensitive display 124 may include additional functionality. Projector screen 122 (e.g., an electronic whiteboard), may receive data from computing device 100 and display the graphical content. In some examples, presence-sensitive display 124 may determine one or more user inputs (e.g., continuous gestures, multi-touch gestures, single-touch gestures, etc.) at projector screen 122 using capacitive, inductive, and/or optical recognition techniques and send indications of such user input using one or more communication units to computing device 100.

FIG. 6 also illustrates mobile device 126 and visual display device 130. Mobile device 126 and visual display device 130 may each include computing and connectivity capabilities. Examples of mobile device 126 may include e-reader devices, convertible notebook devices, hybrid slate devices, etc. Examples of visual display device 130 may include other semi-stationary devices such as televisions, computer monitors, etc. As shown in FIG. 6, mobile device 126 may include a presence-sensitive display 128. Visual display device 130 may include a presence-sensitive display 132. Presence-sensitive displays 128, 132 may include a subset of functionality or all of the functionality of user interface device 12 as described in this disclosure. In some examples, presence-sensitive displays 128, 132 may include additional functionality. In any case, presence-sensitive display 132, for example, may receive data from computing device 100 and display the graphical content. In some examples, presence-sensitive display 132 may determine one or more user inputs (e.g., continuous gestures, multi-touch gestures, single-touch gestures, etc.) at projector screen using capacitive, inductive, and/or optical recognition techniques and send indications of such user input using one or more communication units to computing device 100.

As described above, in some examples, computing device 100 may output graphical content for display at presence-sensitive display 104 that is coupled to computing device 100 by a system bus or other suitable communication channel. Computing device 100 may also output graphical content for display at one or more remote devices, such as projector 120, projector screen 122, mobile device 126, and visual display device 130. For instance, computing device 100 may execute one or more instructions to generate and/or modify graphical content in accordance with techniques of the present disclosure. Computing device 100 may output the data that includes the graphical content to a communication unit of computing device 100, such as communication unit 110. Communication unit 110 may send the data to one or more of the remote devices, such as projector 120, projector screen 122, mobile device 126, and/or visual display device 130. In this way, computing device 100 may output the graphical content for display at one or more of the remote devices. In some examples, one or more of the remote devices may output the graphical content at a presence-sensitive display that is included in and/or operatively coupled to the respective remote devices.

In some examples, computing device 100 may not output graphical content at presence-sensitive display 104 that is operatively coupled to computing device 100. In other examples, computing device 100 may output graphical content for display at both a presence-sensitive display 104 that is coupled to computing device 100 by communication channel 102A, and at one or more remote devices. In such examples, the graphical content may be displayed substantially contemporaneously at each respective device. For instance, some delay may be introduced by the communication latency to send the data that includes the graphical content to the remote device. In some examples, graphical content generated by computing device 100 and output for display at presence-sensitive display 104 may be different than graphical content display output for display at one or more remote devices.

Computing device 100 may send and receive data using any suitable communication techniques. For example, computing device 10 may be operatively coupled to external network 114 using network link 112A. Each of the remote devices illustrated in FIG. 6 may be operatively coupled to network external network 114 by one of respective network links 112B, 112C, and 112D. External network 114 may include network hubs, network switches, network routers, etc., that are operatively inter-coupled thereby providing for the exchange of information between computing device 100 and the remote devices illustrated in FIG. 6. In some examples, network links 112A-112D may be Ethernet, ATM or other network connections. Such connections may be wireless and/or wired connections.

In some examples, computing device 100 may be operatively coupled to one or more of the remote devices included in FIG. 6 using direct device communication 118. Direct device communication 118 may include communications through which computing device 100 sends and receives data directly with a remote device, using wired or wireless communication. That is, in some examples of direct device communication 118, data sent by computing device 100 may not be forwarded by one or more additional devices before being received at the remote device, and vice-versa. Examples of direct device communication 118 may include Bluetooth, Near-Field Communication, Universal Serial Bus, WiFi, infrared, etc. One or more of the remote devices illustrated in FIG. 6 may be operatively coupled with computing device 100 by communication links 116A-116D. In some examples, communication links 116A-116D may be connections using Bluetooth, Near-Field Communication, Universal Serial Bus, infrared, etc. Such connections may be wireless and/or wired connections. As described herein, computing device 100 may be operatively coupled to any other device for the purposes of outputting information (e.g., non-withheld notification information, filtered items, etc.) for delivery (e.g., presentation or display) to the user via any one or more of devices 104, 120, 122, 126, and 130 of FIG. 6.

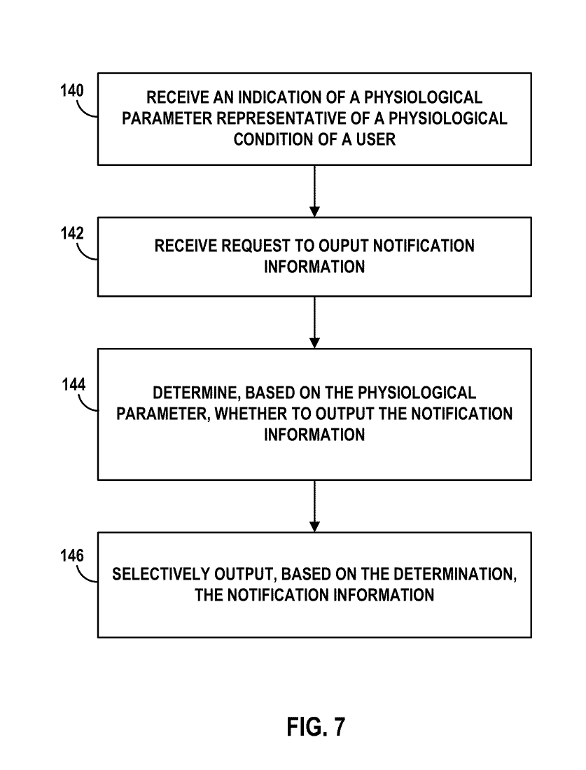

FIG. 7 is a flowchart illustrating example operations of computing device 10 configured to determine whether to output notification information. The processes of FIG. 7 may be performed by one or more processors of a computing device, such as one or more processors 40 of computing device 10 illustrated in FIGS. 1, 2, and 3. For purposes of illustration, FIG. 7 is described within the context of information server system 60, computing device 10, and system 1, illustrated in FIG. 1. Although the technique of FIG. 7 is described with reference to computing device 10 of FIGS. 1, 2, and 3, in other examples, the technique of FIG. 7 may be performed by another computing device or computing system, such as computing device that includes more or fewer components that computing device 10 or mobile computing device 34.

As shown in FIG. 7, context module 24 of computing device 10 may be configured to receive an indication of a physiological parameter representative of a physiological condition of a user (140). For example, processors 40 may receive the indication of the physiological parameter from one of sensors 14. Context module 24 may determine the physiological condition of the user based on the physiological condition and transmit the physiological condition to notification client module 22. Alternatively, notification client module 22 may directly receive the physiological parameter from context module 24 or sensor 14. Notification client module 22 may then receive a request to output notification information from information server system 60 via network 30 (142).

Notification client module 22 may determine, based on the physiological parameter, whether to output, for delivery to the user, the notification information (144). For example, notification client module 22 may withhold at least some notification information when the physiological parameter exceeds a threshold (e.g., the parameter indicates that the user is experiencing a physiological condition in which the notification information should be withheld). Notification client module 22 may selectively output, based on the determination of whether to output the notification information, the notification information (146). In this manner, computing device 10 may control the flow of information delivered to the user, such as reduce or prevent notification information from being delivered to the user during stressful or concentrating situations.

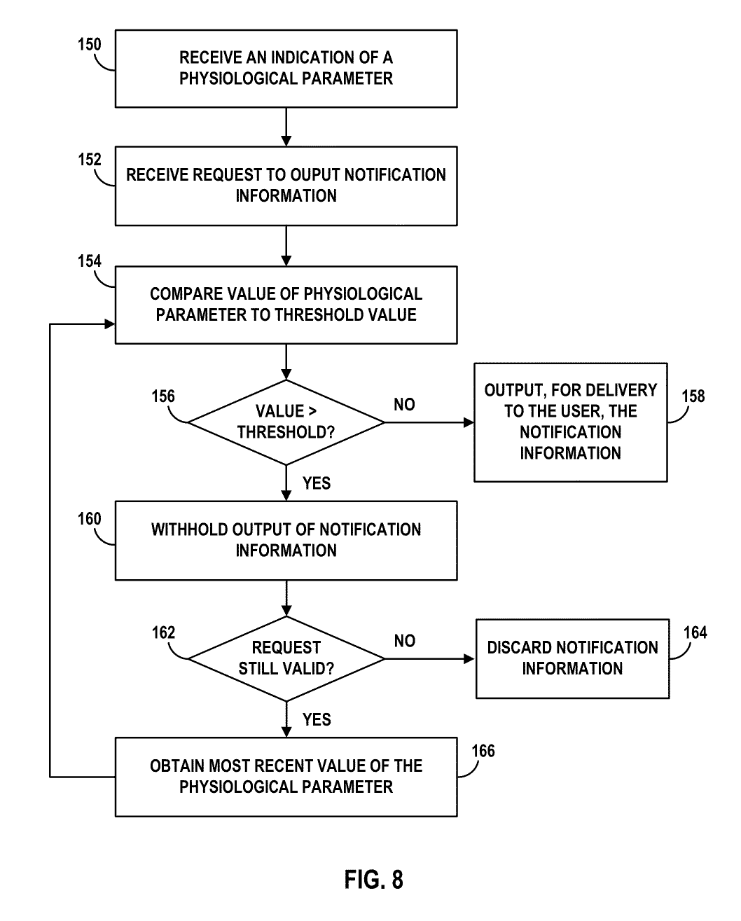

FIG. 8 is a flowchart illustrating example operations of a computing device configured to withhold output of notification information when a value of a physiological parameter exceeds a threshold value. The processes of FIG. 8 may be performed by one or more processors of a computing device, such as one or more processors 40 of computing device 10 illustrated in FIGS. 1, 2, and 3. For purposes of illustration, FIG. 8 is described within the context of information server system 60, computing device 10, and system 1, illustrated in FIG. 1. Although the technique of FIG. 8 is described with reference to computing device 10 of FIGS. 1, 2, and 3, in other examples, the technique of FIG. 8 may be performed by another computing device or computing system, such as computing device that includes more or fewer components that computing device 10 or mobile computing device 34.

As shown in FIG. 8, context module 24 of computing device 10 may be configured to receive an indication of a physiological parameter (e.g., a value of the physiological parameter) representative of a physiological condition of a user (150). For example, processors 40 may receive the indication of the physiological parameter from one of sensors 14. Alternatively, notification client module 22 may directly receive physiological parameter from the respective sensor 14. Notification client module 22 may also receive a request to output notification information from information server system 60 via network 30 (152).

Notification client module 22 may then determine, based on the physiological parameter, whether to output, for delivery to the user, the notification information. Notification client module 22 may compare the value of the physiological parameter to a threshold value (154). The threshold value may be specific for the physiological parameter and, in some examples, specific to the type of notification information, source of the notification information, and/or user associated with the notification information. If notification client module 22 determines that the physiological parameter value is less than the threshold value (“NO” branch of block 156), notification client module 22 may output, for delivery to the user, the notification information (158). If notification client module 22 determines that the physiological parameter value is greater than the threshold value (“YES” branch of block 156), notification client module 22 withholds output of at least some of the notification information (160). For example, the physiological parameter for galvanic skin response exceeding the threshold may be representative of a stressed or concentrated condition of the user. Therefore, some or all of the notification information may be withheld so as not to interrupt the user.

In some examples, notification client module 22 may determine whether notification information should be output based on a single threshold. In other examples, notification client module 22 may utilize two or more threshold values, or two or more ranges of values, to determine which, if any, subsets of notification information is to be withheld from delivery to the user. For example, increasing galvanic skin response (e.g., conductivity due to perspiration) may indicate increasing levels of stress for the user. Notification client module 22 may output both a representation of the notification and the indication of the notification in response to determining that the physiological parameter value is less than both thresholds. Notification client module 22 may output only the representation of the notification and withhold the indication of the notification in response to determining that the physiological parameter value is greater than a first threshold and less than a second threshold. In addition, notification client module 22 may withhold both the representation of the notification and the indication of the notification in response to determining that the physiological parameter value is greater than both thresholds. In this manner, thresholds or rules may be utilized by notification client module 22 selectively withhold, or output, some or all of the received notification information.

Notification client module 22 may then determine if the request to output notification is still valid (e.g., the request has not expired or has been withdrawn) (162). If the request is still valid (“YES” branch of block 162), notification client module 22 may obtain the most recent value of the physiological parameter (166) and again compare the value of the physiological parameter to the threshold value (154). Notification client module 22 may continue this process until notification client module 22 can output the notification information or the request to output the notification information expires or is withdrawn. If the request is no longer valid (e.g., expired or withdrawn) (“NO” branch of block 162), notification client module 22 may discard the notification information (164).

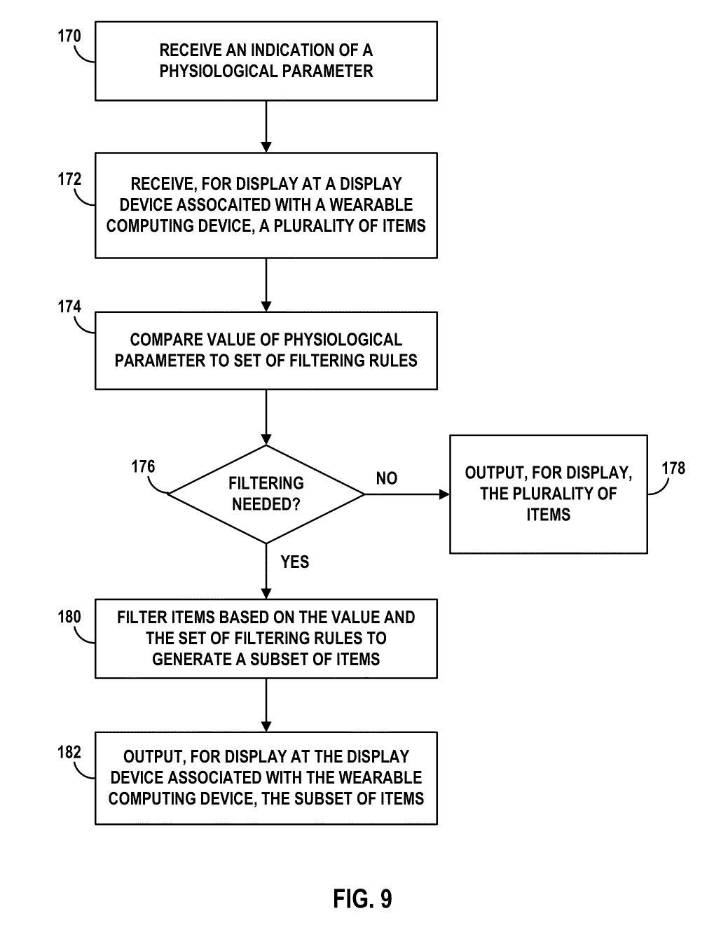

FIG. 9 is a flowchart illustrating example operations of a computing device configured to filter a plurality of items based on a physiological parameter. Computing device 10, for example, may control, based on one or more physiological parameters, the flow of information other than notification information. For example, computing device 10, or any other computing device, may filter a plurality of items based on the detected physiological parameter of the patient. The processes of FIG. 9 may be performed by one or more processors of a computing device, such as one or more processors 40 of computing device 10 illustrated in FIGS. 1, 2, and 3. However, only one processor 40 will be described for purposes of illustration. For purposes of illustration, FIG. 9 is described within the context of information server system 60, computing device 10, and system 1, illustrated in FIG. 1. Although the technique of FIG. 9 is described with reference to computing device 10 of FIGS. 1, 2, and 3, in other examples, the technique of FIG. 9 may be performed by another computing device or computing system, such as a computing device that includes more or fewer components that computing device 10 or mobile computing device 34.

As shown in FIG. 9, context module 24 of computing device 10 may be configured to receive an indication of a physiological parameter (e.g., a value of the physiological parameter) representative of a physiological condition of a user (170). For example, processor 40 may receive the indication of the physiological parameter from one of sensors 14. Alternatively, notification client module 22 may directly receive physiological parameter from the respective sensor 14. Processors 40 may also receive, for display at a display device associated with computing device 10 (e.g., a wearable computing device) a plurality of items (172). The plurality of items may be received from another computing device, such as information server system 60 via network 30, or even generated by computing device 10.

Processor 40 (or a module of computing device 10) may then filter, based on the physiological parameter, the plurality of items to generate a subset of items for display to the user. For example, processor 40 may compare the value of the physiological parameter to a set of filtering rules (174). The filtering rules may instruct processor 40 how to filter the items based on the physiological parameter. For example, the filtering rules may be different for different types of items (e.g., search results or available applications for execution), different sources of the items (e.g., different websites or different services, or even items related to different users (e.g., items contributed from various users). The set of rules may then establish one or more different thresholds for various different items or groups of items.

If processor 40 determines that no filtering is needed (“NO” branch of block 176), processor 40 may output, for display at a display device associated with computing device 10, all of the plurality of items (178). However, if processor 40 determines that filtering does apply to the plurality of items (“YES” branch of block 176), processor 40 may then filter the plurality of items based on the value of one or more respective physiological parameters and the set of filtering rules to generate a subset of items (180). In this manner, the processor 40 may remove one or more of the items to generate the subset of items. Processor 40 may then output, for display at the display device associated with computing device 10, the subset of items.

The process of filtering items may be based on any of the physiological parameters described herein, such as a galvanic skin response, a pulse rate, a breathing rate, or an electrogram. In addition, processor 40 may receive values from two or more different physiological parameters and filter the plurality of items based on each of the values of the respective physiological parameters to generate the subset of the plurality of items. Computing device 10 may compare the values of physiological parameters to value ranges identified by the set of rules. For example, computing device may compare the value of the physiological parameter to a plurality of value ranges for the respective parameter and select one of the plurality of value ranges inclusive of the value of the physiological parameter. In this manner, computing device 10 may select, as the subset of the plurality of items, items associated with the selected one of the plurality of value ranges.

The plurality of items filtered by computing device 10 may include a variety of different items that may or may not be relevant or desired for a user's condition. In one example, the plurality of items comprises a plurality of applications executable by computing device 10 (e.g., a wearable computing device). In another example, the plurality of items may include a set of search results that include one or more actionable links. The search results may be the result of a predictive query input by computing device 10 or another device or in response to a user provided search query. Therefore, computing device 10 may also receive an indication of a search query provided by a user or an automated device. The plurality of items may be generated by information server system 60, for example, in response to the search query and transmitted to computing device 10. In this manner, computing device 10 may filter the plurality of items to generate a smaller set of items relevant to the user experiencing a certain condition. The set of rules used by computing device 10 to filter the items may be directed to filtering certain types of items and/or reducing the number of items output for display to the user.

As described herein, computing device 10 (e.g., a wearable computing device) may perform the filtering process. In other examples, a networked computing device such as information server system 60 may filter the plurality of items and transmit the subset of items back to computing device 10. Computing device 10, or any other device generating the physiological parameters, may transmit the indications of the physiological parameters to information server system 60 via network 30 for filtering. In this manner, the filtering process may be completed by any one or more computing device described herein such that the resulting subset of items can be output and displayed at a display device of a wearable computing device (e.g., user interface device 12 of computing device 10).

Any of the systems (e.g., systems 1 and 2), devices (e.g., computing devices 10 and 34 and information server system 60), and methods described herein may be used within the context of a push notification system and or a pull notification system where information server system 60 transmits notification information to computing device 10 and/or 34. In addition, or alternatively, computing device 10 and/or computing device 34 may execute one or more applications that generate notification information for presentation to the user. In this manner, notification information may be generated or defined locally by computing device 10 or computing device 34 instead of by a remote device (e.g., information server system 60). The systems and devices may, in some examples, utilize push and/or pull notification systems and/or locally generated notification information. For example, computing device 10 may receive notification information pushed from information server system 60 and generate notification information via one or more applications or the operating system executing on computing device 10. Each of the methods for generating and transmitting notification information may be specific to each service, application, device, or system, associated with computing device 10 or 34. For example, an email account may utilize a push notification system and a battery status application executing on computing device 10 may locally generate the notification information.

This disclosure describes various examples and combination of examples. Example 1: a method comprising receiving, by a computing device, an indication of a physiological parameter representative of a physiological condition of a user, receiving, by the computing device, a request to output notification information, determining, by the computing device and based on the physiological parameter, whether to output the notification information, and selectively outputting, by the computing device and based on the determination, the notification information.

Example 2: the method of example 1, wherein the notification information comprises at least one of a representation of a notification and an indication of the notification, and wherein the indication of the notification comprises at least one of a visual alert for the notification, an audible alert for the notification, and a tactile alert for the notification.

Example 3: the method of any of examples 1 and 2, wherein the physiological parameter is at least one of a galvanic skin response, a pulse rate, a breathing rate, and an electrogram.

Example 4: the method of any of examples 1 to 3, wherein the physiological parameter is a first physiological parameter, and wherein the method further comprises receiving an indication of a second physiological parameter representative of the physiological condition of the user, wherein determining whether to output the notification information comprises determining, based on the first physiological parameter and the second physiological parameter, whether to output the notification information.

Example 5: the method of any of examples 1 to 4, wherein the indication comprises a value of the physiological parameter, and wherein the method further comprises comparing the value of the physiological parameter to a threshold value and determining that the value exceeds the threshold value, wherein determining whether to output the notification information comprises, responsive to determining that the value exceeds the threshold value, withholding output of the notification information.

Example 6: the method of example 5, wherein the value is a first value of the physiological parameter, and wherein the method further comprises subsequent to withholding output of the notification information, receiving an indication of a second value of the physiological parameter, determining that the second value is below the threshold value, and responsive to determining that the second value is below the threshold value, outputting the notification information.

Example 7: the method of any of examples 1 to 6, wherein selectively outputting the notification information comprises one of generating a command that instructs a wearable computing device to turn off a display or outputting, for display at a display device associated with a wearable computing device, a graphical user interface without of the notification information.

Example 8: the method of any of examples 1 to 7, wherein the notification information is unrelated to the physiological parameter.

Example 9: the method of any of examples 1 to 8, wherein the notification information is indicative of an event related to at least one application executing on a computing device.

Example 10: the method of any of examples 1 to 9, wherein a wearable computing device comprises the one or more computing devices.

Example 11: the method of any of examples 1 to 10, wherein a networked computing device comprises the one or more computing devices, and wherein receiving the signal comprises receiving, from a wearable computing device and via a network, the indication of the physiological parameter.

Example 12: a computing device comprising at least one processor and at least one module operable by the at least one processor to receive an indication of a physiological parameter representative of a physiological condition of a user, receive a request to output a notification information, determine, based on the physiological parameter, whether to output the notification information, and selectively output, based on the determination, the notification information.

Example 13: the computing device of example 12, wherein the notification information comprises at least one of a representation of a notification and an indication of the notification, and wherein the indication of the notification comprises at least one of a visual alert for the notification, an audible alert for the notification, and a tactile alert for the notification.

Example 14: the computing device of any of examples 12 and 13, wherein the physiological parameter is at least one of a galvanic skin response, a pulse rate, a breathing rate, and an electrogram.

Example 15: the computing device of any of examples 12 to 14, wherein the physiological parameter is a first physiological parameter, and wherein the at least one module is operable by the at least one processor to receive an indication of a second physiological parameter representative of the physiological condition of the user and determine, based on the first physiological parameter and the second physiological parameter, whether to output the notification information.

Example 16: the computing device of any of examples 12 to 15, wherein the indication comprises a value of the physiological parameter, and wherein the at least one module is operable by the at least one processor to compare the value of the physiological parameter to a threshold value, and determine that the value exceeds the threshold value, wherein the at least one module is operable by the at least one processor to determine whether to output the notification information by, responsive to determining that the value exceeds the threshold value, withholding output of the notification information.

Example 17: the computing device of example 16, wherein the value is a first value of the physiological parameter, and wherein the at least one module is operable by the at least one processor to subsequent to withholding output of the notification information, receive an indication of a second value of the physiological parameter, determine that the second value is below the threshold value, and responsive to determining that the second value is below the threshold value, output the notification information.

Example 18: the computing device of any of examples 12 to 17, wherein the at least one module is operable by the at least one processor to selectively output the notification information by generating a command that instructs a wearable computing device to turn off a display or output, for display at a display device associated with a wearable computing device, a graphical user interface without of the notification information.

Example 19: the computing device of any of examples 12 to 18, wherein the notification information is unrelated to the physiological parameter.

Example 20: the computing device of any of examples 12 to 19, wherein the notification information is indicative of an event related to at least one application executing on a computing device.

Example 21: the computing device of any of examples 12 to 20, wherein the computing device comprises a wearable computing device that houses the one or more processors.

Example 22: the computing device of any of examples 12 to 21, wherein the computing device comprises a networked computing device configured to house the one or more processors, and wherein the at least one module is operable by the at least one processor to receive, from a wearable computing device and via a network, the indication of the physiological parameter.

Example 23: a computer-readable storage medium comprising instructions that, when executed, cause one or more processors of a computing device to receive an indication of a physiological parameter representative of a physiological condition of a user, receive a request to output a notification information, determine, based on the physiological parameter, whether to output the notification information, and selectively output, based on the determination, the notification information.

Example 24: a computing device comprising means for performing any of the methods of examples 1-11.

Example 25: a computer-readable storage medium encoded with instructions for causing one or more programmable processors to perform any of the methods recited by examples 1-12.

Example 26: a method comprising receiving, by one or more computing devices, an indication of a physiological parameter representative of a physiological condition of a user, receiving, by the one or more computing devices and for display at a display device associated with a wearable computing device, a plurality of items, filtering, by the one or more computing devices and based on the physiological parameter, the plurality of items to generate a subset of the plurality of items, and outputting, for display at the display device associated with the wearable computing device, the subset of the plurality of items.

Example 27: a method of example 26, wherein the physiological parameter is at least one of a galvanic skin response, a pulse rate, a breathing rate, and an electrogram.

Example 28: a method of any of examples 26 and 27, wherein the indication is a first indication, the physiological parameter is a first physiological parameter, and wherein the method further comprises receiving a second indication of a second physiological parameter representative of the physiological condition of the user, and wherein filtering the plurality of items comprises filtering, based on the first and second physiological parameters, the plurality of items to generate the subset of the plurality of items.

Example 29: a method of any of examples 26 to 28, further comprising comparing a value of the physiological parameter to a threshold value, and determining that the value exceeds the threshold value, wherein filtering the plurality of items comprises filtering, based on the determination, the plurality of items to generate the subset of the plurality of items.

Example 30: a method of any of examples 26 to 29, further comprising comparing the value of the physiological parameter to a plurality of value ranges and selecting one of the plurality of value ranges inclusive of the value of the physiological parameter, wherein filtering the plurality of items comprises selecting, as the subset of the plurality of items, items associated with the selected one of the plurality of value ranges.

Example 31: a method of any of examples 26 to 30, wherein the plurality of items comprises a plurality of applications executable by the wearable computing device.

Example 32: a method of any of examples 26 to 31, wherein the plurality of items comprises a set of search results comprising one or more actionable links.

Example 33: a method of any of examples 26 to 32, further comprising receiving a search query, wherein the plurality of items is generated in response to the search query.

Example 34: a method of any of examples 26 to 33, wherein a wearable computing device comprises the one or more computing devices.

Example 35: a method of any of examples 26 to 34, wherein a networked computing device comprises the one or more computing devices, and wherein receiving the signal comprises receiving, from the wearable computing device and via a network, the signal.

Example 36: a system comprising one or more computing devices configured to perform any of the methods of examples 26-35.

Example 37: a computing device comprising one or more processors configured to perform any of the methods of examples 26-35.

Example 38: a system comprising means for performing any of the methods of examples 26-35.

Example 25: a computer-readable storage medium encoded with instructions for causing one or more programmable processors to perform any of the methods recited by examples 26-35.

In one or more examples, the functions described may be implemented in hardware, software, firmware, or any combination thereof. If implemented in software, the functions may be stored on or transmitted over, as one or more instructions or code, a computer-readable medium and executed by a hardware-based processing unit. Computer-readable media may include computer-readable storage media, which corresponds to a tangible medium such as data storage media, or may include communication media including any medium that facilitates transfer of a computer program from one place to another, e.g., according to a communication protocol. In this manner, computer-readable media generally may correspond to (1) tangible computer-readable storage media, which is non-transitory or (2) a communication medium such as a signal or carrier wave. Data storage media may be any available media that can be accessed by one or more computers or one or more processors to retrieve instructions, code and/or data structures for implementation of the techniques described in this disclosure. A computer program product may include a computer-readable medium.

By way of example, and not limitation, such computer-readable storage media can comprise RAM, ROM, EEPROM, CD-ROM or other optical disk storage, magnetic disk storage, or other magnetic storage devices, flash memory, or any other medium that can be used to store desired program code in the form of instructions or data structures and that can be accessed by a computer. Also, any connection is properly termed a computer-readable medium. For example, if instructions are transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. It should be understood, however, that computer-readable storage media and data storage media do not include connections, carrier waves, signals, or other transient media, but are instead directed to non-transient, tangible storage media. Disk and disc, as used herein, includes compact disc (CD), laser disc, optical disc, digital versatile disc (DVD), floppy disk and Blu-ray disc, where disks usually reproduce data magnetically, while discs reproduce data optically with lasers. Combinations of the above should also be included within the scope of computer-readable media.

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor,” as used herein may refer to any of the foregoing structure or any other structure suitable for implementation of the techniques described herein. In addition, in some aspects, the functionality described herein may be provided within dedicated hardware and/or software modules. Also, the techniques could be fully implemented in one or more circuits or logic elements.

The techniques of this disclosure may be implemented in a wide variety of devices or apparatuses, including a wireless handset, an integrated circuit (IC) or a set of ICs (e.g., a chip set). Various components, modules, or units are described in this disclosure to emphasize functional aspects of devices configured to perform the disclosed techniques, but do not necessarily require realization by different hardware units. Rather, as described above, various units may be combined in a hardware unit or provided by a collection of interoperative hardware units, including one or more processors as described above, in conjunction with suitable software and/or firmware.

Various examples have been described. These and other examples are within the scope of the following claims.

What is claimed is:

1. A method comprising:
receiving, by one or more computing devices and from a sensor, an indication of a physiological parameter representative of a physiological condition of a user;
receiving, by the one or more computing devices and for display at a display device associated with a wearable computing device, a plurality of items;
filtering, by the one or more computing devices and based on the physiological parameter, the plurality of items to generate a subset of the plurality of items; and
outputting, by the one or more computing devices and for display at the display device associated with the wearable computing device, the subset of the plurality of items.

2. The method of claim 1, wherein the physiological parameter comprises at least one of a galvanic skin response parameter, a pulse rate parameter, a breathing rate parameter, or an electrogram parameter.

3. The method of claim 1, wherein the indication is a first indication, the physiological parameter is a first physiological parameter, and wherein the method further comprises receiving a second indication of a second physiological parameter representative of the physiological condition of the user, and wherein filtering the plurality of items comprises filtering, based on the first and second physiological parameters, the plurality of items to generate the subset of the plurality of items.

4. The method of claim 1, further comprising:
comparing a value of the physiological parameter to a threshold value; and
determining that the value exceeds the threshold value, wherein filtering the plurality of items comprises, responsive to determining that the value exceeds the threshold value, filtering, based on the determination, the plurality of items to generate the subset of the plurality of items.

5. The method of claim 1, further comprising:
comparing the value of the physiological parameter to a plurality of value ranges; and
selecting, based on the comparison, one value range of the plurality of value ranges, the selected one value range being inclusive of the value of the physiological parameter, wherein filtering the plurality of items comprises selecting, as the subset of the plurality of items, items associated with the selected one value range of the plurality of value ranges.

6. The method of claim 1, wherein the plurality of items comprises a plurality of applications executable by the wearable computing device.

7. The method of claim 1, wherein the plurality of items comprises a set of search results comprising one or more actionable links.

8. The method of claim 1, further comprising:
receiving, via a user interface of the wearable computing device, a search query; and
transmitting the search query to a networked server; wherein receiving the plurality of items comprises receiving the plurality of items as a set of search results generated by the networked server and in response to the search query.

9. The method of claim 1, wherein the wearable computing device comprises the one or more computing devices.

10. The method of claim 1, wherein a mobile computing device comprises the one or more computing devices, the mobile computing device separate from the wearable computing device, and wherein outputting the subset of the plurality of items comprises outputting, by the mobile computing device, the subset of the plurality of items to the wearable computing device for display.

11. The method claim 1, wherein a networked computing device comprises the one or more computing devices, and wherein receiving the indication of the physiological parameter comprises receiving, from the wearable computing device and via a network, the indication of the physiological parameter.

12. A computing device comprising:
at least one processor;
at least one sensor; and
at least one module operable by the at least one processor to:
receive, from the at least one sensor, an indication of a physiological parameter representative of a physiological condition of a user;
receive, for display at a display device associated with a wearable computing device, a plurality of items;
filter, based on the physiological parameter, the plurality of items to generate a subset of the plurality of items; and
output, for display at the display device associated with the wearable computing device, the subset of the plurality of items.

13. The computing device of claim 12, wherein the at least one sensor comprises a galvanic skin response sensor.

14. The computing device of claim 12, wherein the at least one sensor comprises a heart rate sensor.

15. The computing device of claim 12, wherein the at least one sensor comprises a blood pressure sensor.

16. The computing device of claim 12, wherein the at least one module is operable by the at least one processor to:
compare a value of the physiological parameter to a threshold value;
determine that the value exceeds the threshold value; and
responsive to determining that the value exceeds the threshold value, filter, based on the determination, the plurality of items to generate the subset of the plurality of items.

17. The computing device of claim 12, wherein the at least one module is operable by the at least one processor to:
compare the value of the physiological parameter to a plurality of value ranges;
select, based on the comparison, one value range of the plurality of value ranges, the selected one value range being inclusive of the value of the physiological parameter; and
select, as the subset of the plurality of items, items associated with the selected one value range of the plurality of value ranges.

18. The computing device of claim 12, wherein the plurality of items comprises a plurality of applications executable by the wearable computing device.

19. The computing device of claim 12, wherein the wearable computing device comprises the computing device.

20. A computer-readable storage medium encoded with instructions that, when executed, cause one or more programmable processors to:
receive, from a sensor, an indication of a physiological parameter representative of a physiological condition of a user;
receive, for display at a display device associated with a wearable computing device, a plurality of items;
filter, based on the physiological parameter, the plurality of items to generate a subset of the plurality of items; and
output, for display at the display device associated with the wearable computing device, the subset of the plurality of items.
